(19) United States
(12) Patent Application Publication
(10) Pub. No.: US 2026/0102526 A1
(43) Pub. Date: Apr. 16, 2026

(54) 177LUTETIUM-DOTA-TATE FORMULATIONS

(71) Applicant: Curium US LLC, St. Louis, MO (US)

(72) Inventors: Amanda Donovan, St. Louis, MO (US); Allan Casciola, St. Louis, MO (US); Chris Vascoe, St. Louis, MO (US); David Pipes, St. Louis, MO (US)

(21) Appl. No.: 19/251,254

(22) Filed: Jun. 26, 2025

Related U.S. Application Data
(60) Provisional application No. 63/665,007, filed on Jun. 27, 2024.

(51) Int. Cl. A61K 51/08; A61K 47/10; A61K 47/18; A61K 47/22; A61P 35/00

(52) U.S. Cl. CPC A61K 51/088; A61K 47/10; A61K 47/183; A61K 47/22; A61K 51/083; A61P 35/00; A61K 2121/00

(57) ABSTRACT

The present disclosure relates to concentrated and stable parenteral ¹⁷⁷Lutetium-DOTA-TATE radionuclide complex solutions. Drug stability is achieved by the combination of from 5 mg/mL to 25 mg/mL of ascorbic acid or a salt thereof and from 1 v/v % to 3 v/v % ethanol.

### CROSS-REFERENCE TO RELATED APPLICATION

This application claims the benefit of U.S. Provisional Patent Application No. 63/665,007 filed on 27 Jun. 2024 which is hereby expressly incorporated by reference in its entirety.

### BACKGROUND

The field of the disclosure relates generally to ¹⁷⁷Lu-DOTA-TATE formulations for radionuclide therapy.

¹⁷⁷Lu-DOTA-TATE drug formulations are useful for the treatment of cancers that express somatostatin receptors.

Problematically, radiopharmaceuticals are unstable and the radiochemical purity can fall to less than 95% after hours or a few days. This results in various challenges regarding the manufacture, storage and transport of radiopharmaceuticals, such as ¹⁷⁷Lu-DOTA-TATE drug formulations. Therefore, for the application of radiopharmaceuticals only a small window is available after manufacturing.

In order to reduce this problem, antioxidants, such as gentisic acid, ethanol, ascorbic acid, histidine, melatonin, methionine, and S-methionine may be added. However, for peptides labeled with ¹⁷⁷Lu, such as DOTA-TATE (oxodotreotide), often complex mixtures of antioxidants or specific time points of their addition are required to obtain the desired effect over an acceptable period of time. For example, Maus et al. reports the addition of ascorbic acid after radiolabeling and purification of ¹⁷⁷Lu-DOTA-TATE (See Stephan Maus et al., Aspects on radiolabeling of 177Lu-DOTA-TATE: After C18 purification re-addition of ascorbic acid is required to maintain radiochemical purity, International Journal of Diagnostic Imaging, 2014, Vol. 1, No. 1). Further, U.S. Pat. No. 10,756,278 B2 requires a complex mixture of gentisic acid, ascorbic acid, and EDTA after radiolabeling to obtain the desired stability against radiolysis of 95% at 72 hours after synthesis. De Blois et al. and Breeman et al. even suggest the addition of a mixture of 50 mM ascorbic acid, 10% (v/v) ethanol and 50 mM L-methionine (See Erik de Blois et al. Effectiveness of Quenchers to Reduce Radiolysis of (¹¹¹)In or (¹⁷⁷)Lu-labeled Methionine-Containing Regulatory Peptides. Maintaining Radiochemical Purity as Measured by HPLC, Curr Top Med Chem. 2012; 12(23):2677-85; see also Wouter A. P. Breeman, Practical Aspects of labeling DTPA- and DOTA-Peptides with ⁹⁰Y, ¹¹¹In, ¹⁷⁷Lu, and ⁶⁸Ga for Peptide-Receptor Scintigraphy and Peptide-Receptor Radionuclide Therapy in Preclinical and Clinical Applications, The University of New Mexico Health Sciences Center, VOLUME 16, LESSON 5: 11/16/2012). According to these reports, for ¹⁷⁷Lu-labeled peptides, the stabilizer, or the second or third stabilizing component, is added only after radiolabeling in acetate or HEPES buffer.

Problematically, drug products containing ethanol stabilizer in excess of above 5 v/v % may be associated with tolerability issues. Further problematically, stabilizers may have a negative impact on the complexation of the radionuclide into the chelating agent and/or may have a limited solubility and precipitate from the drug formulation solution.

A need therefore exists for ¹⁷⁷Lu-DOTA-TATE drug formulations which can be: (i) produced at commercial scale; (ii) delivered as a sufficiently stable and sterile solution; (iii) have a sufficiently high concentration of ¹⁷⁷Lu-DOTA-TATE to provide for a small infusion volume; and (iv) which has high physiological tolerability.

### BRIEF DESCRIPTION

In some aspects, the present disclosure is directed to a parenteral ¹⁷⁷Lu-DOTA-TATE drug formulation comprising: (1) a therapeutically effective amount of a ¹⁷⁷Lu-DOTA-TATE complex; (2) a stabilizer component comprising ascorbic acid, or a salt thereof, and ethanol wherein the concentration of ascorbic acid or a salt thereof is from 5 mg/mL to 25 mg/mL, and the concentration of ethanol is from 1 v/v % to 3 v/v % ethanol; and (3) water.

In some aspects, the present disclosure is directed to a parenteral ¹⁷⁷Lu-DOTA-TATE drug formulation consisting essentially of: (1) a therapeutically effective amount of a ¹⁷⁷Lu-DOTA-TATE complex; (2) ascorbic acid, or a salt thereof, at a concentration of from 5 mg/mL to 25 mg/mL; (3) ethanol at a concentration of from 1 v/v % to 3 v/v %; (4) diethylenetriamine pentaacetic acid (DTPA); and (5) water.

In some aspects, the present disclosure is directed to a parenteral ¹⁷⁷Lu-DOTA-TATE drug formulation consisting of: (1) a therapeutically effective amount of a ¹⁷⁷Lu-DOTA-TATE complex; (2) ascorbic acid, or a salt thereof, at a concentration of from 5 mg/mL to 25 mg/mL; (3) ethanol at a concentration of from 1 v/v % to 3 v/v %; (4) diethylenetriamine pentaacetic acid (DTPA); and (5) water.

In some aspects, the parenteral ¹⁷⁷Lu-DOTA-TATE drug formulations of the present disclosure provide for a radiochemical purity of not less than 95% 72 hours after formulation thereof.

In some aspects, the parenteral ¹⁷⁷Lu-DOTA-TATE drug formulations of the present disclosure provide for a radiochemical purity of not less than 95% up to 7 days after formulation thereof.

In some aspects, the present disclosure is directed to a method of treating cancers that express somatostatin receptors, the method comprising administering to a subject in need thereof a therapeutically effective amount of a ¹⁷⁷Lu-DOTA-TATE drug formulation of the disclosure.

In some aspects, the present disclosure is directed to a process for preparing a ¹⁷⁷Lu-DOTA-TATE drug formulation comprising the following order of steps. In a first step, prepare ¹⁷⁷Lu-DOTA-TATE in a radiolabeling step comprising the following order of steps: (i) in a reactor combine, in any order, ¹⁷⁷LuCl₃, DOTA-TATE in solution in a reaction buffer comprising the stabilizer sodium ascorbate, and additional reaction buffer comprising the stabilizer sodium ascorbate, (ii) preheat the reactor contents to a radiolabeling reaction temperature, and (iii) react the reactor contents at the radiolabeling reaction temperature for a reaction time to form a solution of radiolabeled ¹⁷⁷Lu-DOTA-TATE. In a second step, combine the solution of radiolabeled ¹⁷⁷Lu-DOTA-TATE with dilution buffer comprising the stabilizers sodium ascorbate and ethanol and the chelator diethylenetriamine pentaacetic acid (DTPA) to form bulk ¹⁷⁷Lu-DOTA-TATE drug product at the desired radioactivity concentration. The solution of radiolabeled ¹⁷⁷Lu-DOTA-TATE of the first step is combined with the dilution buffer in the second step in the absence of any intervening process steps. In a third step, sterile filter the bulk ¹⁷⁷Lu-DOTA-TATE drug product and dispense into single-use vials. The solution of radiolabeled ¹⁷⁷Lu-DOTA-TATE of the first step is combined with the dilution buffer in the second step in the absence of any intervening process steps.

### BRIEF DESCRIPTION OF THE DRAWINGS

FIG. 1 is a plot of K_{d} (equilibrium disassociation constant) and Bₘₐₓ (receptor density) for a composition of the present disclosure and a prior composition.

FIG. 2 is a plot of specific internalized activity for a composition of the present disclosure and a prior art composition.

FIG. 3 is a depiction of an infusion method.

FIG. 4 is an HRMS spectrum for Lu-DOTA-TATE.

FIG. 5 is an HPLC chromatogram of a Lu-DOTA-TATE reference standard.

FIG. 6 is an HPLC chromatogram of a DOTA-TATE reference standard.

FIG. 7 is an HPLC chromatogram of a ¹⁷⁷Lu-DOTA-TATE drug product of the present disclosure.

FIG. 8 is a depiction of indirect and direct routes for the preparation of ¹⁷⁷Lu.

FIG. 9 is a plot of % radiochemical purity as a function of time for ¹⁷⁷Lu-DOTA-TATE drug formulations comprising: 2.5 mg/mL of the anti-oxidant sodium ascorbate; 2.5 mg/mL of the anti-oxidant sodium ascorbate and 1.5 v/v % of the anti-oxidant ethanol; 20 mg/mL of the anti-oxidant sodium ascorbate; and 20 mg/mL of the anti-oxidant sodium ascorbate and 1.5 v/v % of the anti-oxidant ethanol.

FIG. 10 is a plot of % radiochemical purity as a function of time (0 days, 3 days, and 7 days) for ¹⁷⁷Lu-DOTA-TATE drug formulations comprising: 3 v/v % ethanol and 10 mg/mL sodium ascorbate; 1.5 v/v % ethanol and 10 mg/mL sodium ascorbate; 6 v/v % ethanol and 20 mg/mL sodium ascorbate; 6 v/v % ethanol and 10 mg/mL sodium ascorbate; and 1.5 v/v % ethanol and 20 mg/mL sodium ascorbate.

FIGS. 11A to 11E are depictions of HPLC chromatograms for ¹⁷⁷Lu-DOTA-TATE compositions of the present disclosure comprising: 1.5 v/v % ethanol and 10 mg/mL sodium ascorbate (FIG. 11A); 3 v/v % ethanol and 10 mg/mL sodium ascorbate (FIG. 11B); 6 v/v % ethanol and 10 mg/mL sodium ascorbate (FIG. 1 IC); 6 v/v % ethanol and 20 mg/mL sodium ascorbate (FIG. 11D); and 1.5 v/v % ethanol and 20 mg/mL sodium ascorbate (FIG. 11E).

FIG. 12 is a process flow diagram for the preparation of ¹⁷⁷Lu-DOTA-TATE according to one embodiment of the disclosure.

### DETAILED DESCRIPTION

In accordance with the disclosure, a ¹⁷⁷Lu-DOTA-TATE drug formulation stabilizer has been discovered comprising, consisting essentially of, or consisting of the selection and combination of the components: (i) sodium ascorbate at a concentration in the ¹⁷⁷Lu-DOTA-TATE drug formulation of from 10 mg/mL to 30 mg/mL; and (ii) ethanol at a concentration in the ¹⁷⁷Lu-DOTA-TATE drug formulation of from 1 v/v % to 3 v/v %. Said stabilizer effectively provides for concentrated, stable, and tolerable ¹⁷⁷Lu-DOTA-TATE drug formulations.

In some embodiments, the radiochemical purity of the ¹⁷⁷Lu-DOTA-TATE drug formulation is not less than 95% at 72 hours after formulation thereof.

In some embodiments, the radiochemical purity of the ¹⁷⁷Lu-DOTA-TATE drug formulation is not less than 95% up to 7 days after formulation thereof.

In some embodiments, the content of ¹⁷⁷Lu-DOTA-TATE complex in the ¹⁷⁷Lu-DOTA-TATE drug formulation is from 222 mCi to 272 mCi.

In some embodiments, the amount of unbound ¹⁷⁷Lu in the ¹⁷⁷Lu-DOTA-TATE drug formulation is not more than 1%.

### Definitions

As used herein, “K_{d}” refers to the radioligand equilibrium disassociation constant and is a measure of affinity, and is one measure of specific biological activity of drugs and hormones. See, for instance, Pedersen, J B and Lindup, W E, Interpretation and analysis of receptor binding experiments which yield non-linear Scatchard plots and binding constants dependent upon receptor concentration, Biochem Pharmacol, 1994 Jan. 20; 47(2), the contents of which are incorporated herein in its entirety. See, further, Wiener, H L and Reith, M E, Determination of radioligand specific activity using competition binding assays, Anal. Biochem. 1992 Nov. 15; 207(1):58-62, the contents of which are incorporated herein in its entirety. See further, Behnammanesh, Hossein, et al., Preclinical study of a new 177Lu-labeled somatostatin receptor antagonist in HT-29 human colorectal cells, Asia Ocean J Nucl Med Biol, 2020 Spring; 8(2): 109-115, the contents of which are incorporated herein in its entirety.

As used herein, “Bₘₐₓ” refers to the maximal number of binding sites (receptor density) and is a measure of the capacity of a receptor preparation for various ligands. See, for instance, Pedersen, J B, et al., Weiner H L, et al., and Behnammanesh, Hossein, et al.

As used herein, “specific internalized activity” refers to an expression, as a percentage, of the cell activity relative to the specific cell-bound activity. See, for instance, Vizquez, S. M, et al., Translational Development of a Zr-89-Labeled Inhibitor of Prostate-specific Membrane Antigen for PET Imaging in Prostate Cancer, Mol Imaging Biol 24, 115-125 (2022), the contents of which are incorporated herein in its entirety.

As used herein, a “somatostatin receptor” refers to receptors for the ligand somatostatin, which is a small neuropeptide associated with neural signaling, particularly in the post-synaptic response to NMDA receptor co-stimulation/activation. See, for instance, Lou L., Principles of Neurobiology, New York, NY: Garland Science, Taylor and Francis Group. pp. 109-110 (2016). Five somatostatin receptors are known including: SST₁ (SSTR1); SST₂ (SSTR2); SST₃ (SSTR3); SST₄ (SSTR4); and SST₅ (SSTR5). See Hoyer D., et al., Classification and nomenclature of somatostatin receptors, Trends Pharmacol. Sci. 16 (3): 86-8 (2015), the contents of which are incorporated herein in its entirety.

As used herein, a “therapeutically effective amount” refers to an amount of the ¹⁷⁷Lu-DOTA-TATE drug formulations of the present disclosure that (i) treats cancers that express somatostatin receptors, (ii) attenuates, ameliorates, or eliminates one or more symptoms of cancers that express somatostatin receptors, or (iii) prevents or delays the onset of one or more symptoms of cancers that express somatostatin receptors. The therapeutically effective amount of the ¹⁷⁷Lu-DOTA-TATE drug formulations may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. For cancer therapy, efficacy of ¹⁷⁷Lu-DOTA-TATE can be measured, for example, by assessing the time to disease progression and/or determining the response rate.

As used herein, the term “salts” is meant to include salts of the referenced compound which are prepared with pharmaceutically acceptable acids or bases. Non-limiting examples of suitable acids and bases include NaOH, KOH, NH₄OH, HCl, H₂SO₄, and organic acids (for instance malic acid).

As used herein, the term “purity”, unless otherwise indicated, refers to the amount of a compound in a sample as compared to the total amount of compounds in the sample. In some aspects, purity may be measured by high pressure liquid chromatography (HPLC) analysis where the area % a product represents purity.

As used herein, the term “parenteral” refers to administration of ¹⁷⁷Lu-DOTA-TATE formulations of the present disclosure by injection or infusion, such as intravenously.

Where an embodiment or a portion thereof is defined with an open-ended term such as “comprising,” it should be readily understood that (unless otherwise stated) the description should be interpreted to also describe such an embodiment using the terms “consisting essentially of” or “consisting of”.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claims.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

As used herein, the indefinite articles “a” and “an” preceding an element or component of the disclosure are intended to be nonrestrictive regarding the number of instances (i.e., occurrences) of the element or component. Therefore “a” or “an” should be read to include one or at least one, and the singular word form of the element or component also includes the plural unless the number is obviously meant to be singular.

Unless defined otherwise, all terms and phrases used herein include the meanings that the terms and phrases have attained in the art, unless the contrary is clearly indicated or clearly apparent from the context in which the term or phrase is used. Although any methods and materials similar or equivalent to those described herein can be used in the practice or testing of the present disclosure, particular methods and materials are now described.

### ¹⁷⁷Lu-DOTA-TATE

177 Lutetium DOTA-TATE (¹⁷⁷Lu-DOTA-TATE) is a chelated complex of the 177 isotope of lutetium that is useful in peptide receptor radionuclide therapy for the treatment of cancers that express somatostatin receptors. See, for instance, Wang L., et al., Somatostatin receptor-based molecular imaging and therapy for neuroendocrine tumors, BioMed Research International, 2013: 102819, the contents of which are incorporated herein in its entirety. In some embodiments, the cancer is a neuroendocrine cancer. In some embodiments, the somatostatin receptor is selected from SSTR1, SSTR2, SSTR3, SSTR4, SSTR5, and combinations thereof. In some embodiments, the somatostatin receptor is SSTR2.

The chemical name for ¹⁷⁷Lu-DOTA-TATE is [¹⁷⁷Lu]lutetium-N-[(4,7,10-tricarboxymethyl-1,4,7,10-tetraazacyclododec-1-yl)acetyl]-D-phenylalanyl-L-cysteinyl-L-tyrosyl-D-tryptophanyl L-lysyl-L-threoninyl-L-cysteinyl-L-threonin-cyclic(2-7)disulfide. The structure of ¹⁷⁷Lu-DOTA-TATE is as follows:

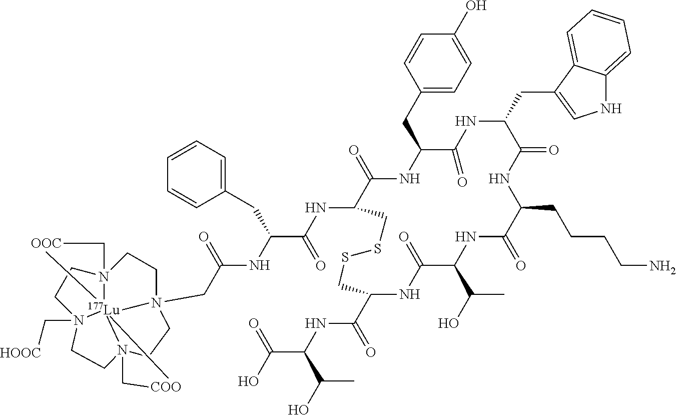

See, for instance, Hennrich U. and Kopka K., Lutathera®: The first FDA-and EMA-approved radiopharmaceutical for peptide receptor radionuclide therapy, Pharmaceuticals. 2019; 12(3), the contents of which are incorporated herein in its entirety.

LUTATHERA® is a ¹⁷⁷Lu-DOTA-TATE listed drug product (NDA 208700). See Center for Drug Evaluation and Research (CDER), Application Number: 2087000rig1s000 Product Quality Review(s).; 2017, the contents of which are incorporated herein in its entirety.

### DOTA-TATE

DOTA-TATE is a cyclic peptide containing 8-amino acids with a terminal peptide bond to the chelate DOTA. DOTA is 1,4,7,10-tetraazacyclododecane-1,4,7,10-tetraacetic acid. The DOTA-chelate is attached to the D-Phe end of the peptide through an amide linkage by utilizing one of the carboxylic acid groups in the ligand, and the free amino group of H₂N-D-Phe-Cys-Tyr-D-Trp-Lys-Thr-Cys-Thr (cyclo 2,7). D-Trp4 and Lys5 each possess an N-atom that can be protonated.

The 8-amino acid, cyclic structure with the DOTA chelate attached at the phenylalanine peptide is as follows:

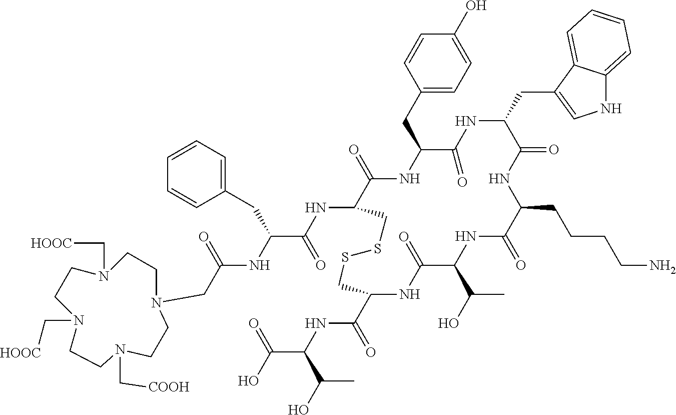 [SMILES: CC(O)[C@H](NC(=O)[C@@H]1CSSC[C@H](NC(=O)[C@@H](Cc2ccccc2)NC(=O)CN2CCN(CC(=O)O)CCN(CC(=O)O)CCN(CC(=O)O)CC2)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

DOTA-TATE (TFA salt) is a peptide that is cyclized through a disulfide bridge between the SH groups of the cysteine residues. The counter ion of the molecule is trifluoroacetic acid (TFA). The precursor exhibits stereoisomerism due to the presence of ten (10) chiral centers. Enantiomeric purity is controlled via the use of stereospecific building blocks and is ultimately monitored via GC-MS (Ph. Eur.) as described in the starting material specification. Polymorphism is not considered relevant since DOTA-TATE is fully dissolved prior to incorporation into the drug substance manufacturing process. See European Medicines Agency, Assessment Report-Lutathera; 2017, the contents of which are incorporated herein in its entirety.

¹⁷⁷LuCl₃

¹⁷⁷LuCl₃ is the radioactive chemical precursor for the drug products and formulations of the disclosure.

It is believed that lutetium has the electronic configuration [Xe]4f¹⁴5d¹6s² with a fully filled 4f-orbital. It is further believed that the Lu³⁺ ion is formed by the loss of the two 6 s-electrons and one 5d-electron and has an electronic configuration of [Xe]4f¹⁴. Lutetium exclusively exists in the +3 oxidation state, with said ion designated as [¹⁷⁷Lu]Lu³⁺ (or ¹⁷⁷Lu³⁺), which precludes any solution chemistry reduction-oxidation complications and commonly forms nine coordination complexes. This translates to the formation of a single molecular entity on complexation with an appropriate ligand (except for the possibility of stereoisomers).

Lu is also strongly electropositive, meaning that Lu³⁺ will strongly attract electronegative ions (e.g., O— and N— atoms of simple non-cyclic and multidentate ligands) thereby providing the basis for complexation of ¹⁷⁷Lu³⁺ with the DOTA chelate present on the DOTA⁰-Tyr³-octreotate ligand.

Lutetium chloride is a colorless or white monoclinic crystal soluble having aqueous solubility and a non-chiral molecular structure. Polymorphism has not been observed. See European Medicines Agency, Scientific Discussion, Published 2004, at https://www.ema.europa.eu/en/documents/scientific-discussion/datscan-epar-scientific-discussion_en.pdf, the contents of which are incorporated herein in its entirety. Based on the chemical form, radiolysis of [¹⁷⁷Lu]LuCl₃ is not expected. See European Medicines Agency. Lutetium-177 decays with a half-life of 6.647 days to the ground state of ¹⁷⁷Hf. During these radioactive decay events, ¹⁷⁷Lu emits β-particles with an Eβ(max) of 497 keV (79.3%), 384 keV (9.1%) and 176 keV (12.2%) and low-energy gamma photons [Eγ=113 keV (6.6%), 208 keV (11%)].

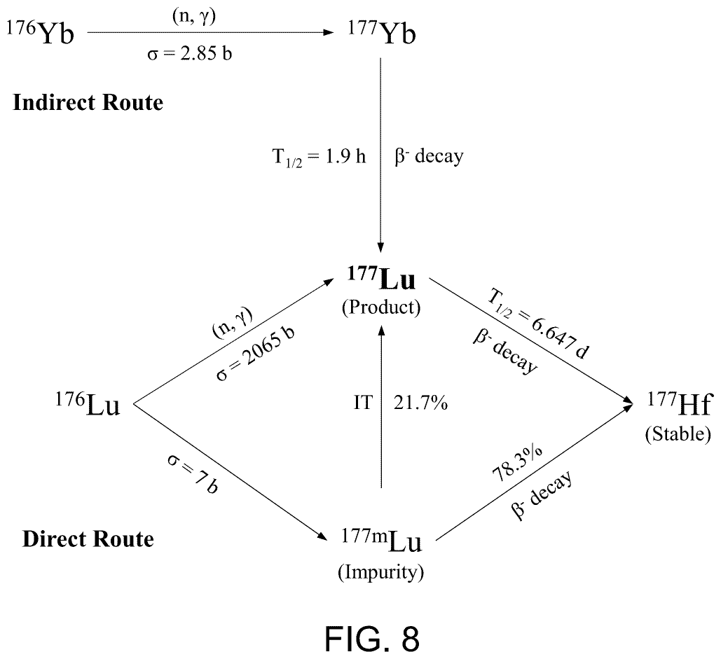

¹⁷⁷Lu³⁺ has a theoretical maximum specific activity of 4.10 TBq/mg or 110 Ci/mg. As depicted in FIG. 8, there are two ways of producing ¹⁷⁷Lu within the scope of the disclosure: (1) a direct route which involves the irradiation of a ^{175/176}Lu target and (2) an indirect route which involves the irradiation of a ¹⁷⁶Yb target which subsequently decays to ¹⁷⁷Lu³⁺. The details of each production route including the empirical specific activity achieved with each production method are summarized in Table A below where “SA” refers to specific activity, “RNP” refers to radionuclidic purity, and “PRRT” refers to peptide receptor radionuclide therapy.

TABLE A

| | Nuclear | Specific | |
|---|---|---|---|
| Route | Reaction | Activity | Comments |
| Direct | ¹⁷⁶Lu(n,γ)¹⁷⁷Lu | 740-1110 | 25% of atoms are ¹⁷⁷Lu, 75% consist of non- |
| | | GBq/mg | radioactive ^{175/176}Lu. |
| | | | SA is sufficient for PRRT but the shelf-life of ¹⁷⁷Lu is |
| | | | limited for PRRT and for use in applications that may |
| | | | require higher SA. See Dash A, Pillai MRA, and |
| | | | Knapp FF, Production of ¹⁷⁷Lu for Targeted |
| | | | Radionuclide Therapy: Available Options. Nucl Med |
| | | | Mol Imaging, 2015; 49(2): 85-107, the contents of |
| | | | which are incorporated herein in its entirety. |
| | | | ^{177m}Lu is produced as a by-product. |
| Indirect | ¹⁷⁶Yb(n,γ)¹⁷⁷Yb | >2.96 | High RNP ¹⁷⁷Lu. |
| | [symbol] ¹⁷⁷Lu | TBq/mg | ¹⁷⁷Lu has longer shelf life since there is no appreciable |
| | | | decrease in SA. |

In some embodiments, ¹⁷⁷LuCl₃ may be produced by a carrier-free route involving neutron bombardment (n, γ) of a highly enriched ¹⁷⁶Yb (≥98%) oxide material to produce ¹⁷⁷Yb with a half-life of 1.9 hours, which then undergoes beta decay to produce ¹⁷⁷Lu. In such embodiments, the decay mode, photon, and half-life characteristics are as follows. Decay mode: 100% beta, daughter of ¹⁷⁷Hf. Photons: 112.95 keV (6.23% abundance); 208.37 keV (10.41% abundance). Half-life: 6.647 days. The energies of the two most prominent radionuclide impurities (i.e., ^{177m}Lu and ¹⁷⁵Yb) range from 113 keV to 419 keV. ¹⁶⁹Yb and ¹⁶⁹Lu impurities are produced from the bombardment of ¹⁶⁸Yb, making up a small fractional abundance in the enriched ¹⁷⁶Yb oxide starting material. ¹⁶⁹Lu identifying photo emissions expand the energy range to about 1000 keV.

In some embodiments of the disclosure, ¹⁷⁷Lu-DOTA-TATE compositions are prepared using ¹⁷⁷Lu³⁺ produced via direct methods.

In some embodiments of the disclosure, ¹⁷⁷Lu-DOTA-TATE compositions are prepared using ¹⁷⁷Lu³⁺ produced via indirect methods.

In some embodiments of the disclosure, ¹⁷⁷Lu-DOTA-TATE compositions are prepared using non-carrier added (NCA) ¹⁷⁷Lu³⁺.

In some embodiments of the disclosure, ¹⁷⁷Lu-DOTA-TATE compositions are prepared using non-carrier added (NCA) ¹⁷⁷Lu³⁺ produced via indirect methods. In such embodiments, it is believed that the drug product and formulations will have higher specific activity since there is less non-radioactive ¹⁷⁵Lu³⁺ available to form M-DOTA-TATE species. Furthermore, it is believed that the radionuclidic impurity profile will be different since ^{177m}Lu³⁺ is not a by-product of the indirect production method and therefore will not be present in the proposed drug formulation.

In any of the various embodiments of the disclosure for the preparation of ¹⁷⁷Lu³⁺, ¹⁷⁷Lu³⁺ can be isolated from the parent isotope and other radionuclidic impurities by radiochemical separation methods known in the art. See, for instance, Salek N, Shamsaei M, Maragheh M G, Arani S S, and Samani A B, Production and quality control 177Lu (NCA)-DOTMP as a potential agent for bone pain palliation, J Appl Clin Med Phys. 2016; 17(6):128-139, the contents of which are incorporated herein in its entirety.

In some embodiments of the disclosure, the specification limits for ¹⁷⁷LuCl₃ are as indicated in Tables B and C below where “LSL” refers to lower specification limit, “USL” refers to upper specification limit, and “NLT” refers to not less than. The limits in Table B are based on the Ph. Eur. Monograph, Lutetium (¹⁷⁷Lu) solution for Radiolabelling, the contents of which are incorporated herein in its entirety.

TABLE B

| Variable | Units | LSL | USL |
|---|---|---|---|
| Cu | μg/GBq | 0 | 1.0 |
| Fe | μg/GBq | 0 | 0.5 |
| Pb | μg/GBq | 0 | 0.5 |
| Zn | μg/GBq | 0 | 1.0 |
| Yb | μg/GBq | 0 | 0.1 |

TABLE C

| Attribute | Specification Limit |
|---|---|
| pH | 1.0-2.0 |
| Radiochemical Identity | Lu-177 confirmed by TLC (R_{f} = 0.4-0.7) |
| Radiochemical Purity | NLT 99% of total radioactivity due to |
| | ¹⁷⁷Lu³⁺ |
| Radioactivity Concentration | Between 33-81 GBq/mL |

Based on the electronic configuration of Lu³⁺, the chemical bonding in Lu³⁺-DOTA⁰-Tyr³-Octreotate is considered ionic. Despite this, the bonding in these complexes does not fit the classical definition of a “salt,” but rather, metal-ligand coordination complexes (charged and uncharged) of Lu³⁺ are believed to exist as stable discrete molecular entities, with well-defined geometry. See Hennrich, et al.

In general, metals from the lanthanide group can have large coordination numbers (up to 12). It is believed that lutetium (¹⁷⁷Lu³⁺) utilizes all of the possible coordination sites to coordinate to all four (4) ring N-atoms, and the three (3) available carboxylic acid arms (O-atoms) of DOTA. Based on X-ray single-crystal analysis for Lu-DOTA, it is believed that the bifunctional chelator DOTA acts as an octadentate chelator and demonstrates similar structural properties to DOTA complexes reported incorporating lighter lanthanide ions. See Aime S, Barge A, Botta M, Fasano M, Ayala J D, and Bombieri G., Crystal structure and solution dynamics of the lutetium(III) chelate of DOTA, Inorganica Chim Acta. 1996; 246(1-2 SPEC. ISSUE):423-429, the contents of which are incorporated herein in its entirety.

Once coordinated, the ¹⁷⁷Lu³⁺ ion is buried in the complex, with the metal ion flanked by the four (4)N-atoms (below the plane of the paper) and the three (3) carboxylic acid groups (above plane). Since the three (3) carboxylic acid groups of DOTA (each having a −1 charge) are involved in binding to ¹⁷⁷Lu³⁺, the complex carries no charge (i.e., +3−3=0). The molecular weight of ^{nat}Lu-DOTA⁰-Tyr3-Octreotide is 1609.6 g/mol. See Center for Drug Evaluation and Research (CDER).

### Characterization of ¹⁷⁷Lu-DOTA-TATE

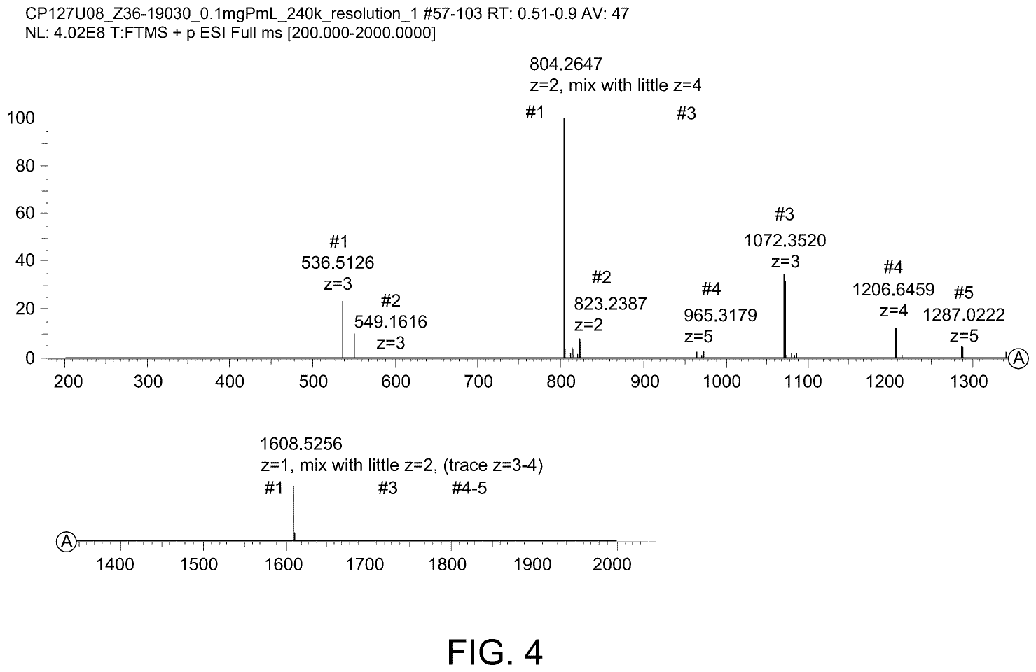

In any of the various embodiments of the disclosure, ¹⁷⁷Lu-DOTA-TATE samples may be characterized by a non-radioactive reference standard prepared using non-radioactive ^{nat}Lu-DOTA-TATE which may then be characterized by high-resolution mass spectrometry (HRMS), infrared spectroscopy, and high performance liquid chromatography (HPLC). A HRMS spectrum is depicted in FIG. 4 and the signals observed are assigned as per the following Table D.

TABLE D

| | Mass | Theoretical | |
|---|---|---|---|
| Species | (Deconvoluted) | (Expected Mass) | ID |
| 1 | 1606.515 | 1606.512 | Monomer |
| 2 | 1644.463 | 1644.468 | Monomer with |
| | | | adduct ion |
| | | | (K⁺ ion replated |
| | | | with H⁺ ion) |
| 3 | 3213.032 | 3213.024 | Dimer |
| 4 | 4819.548 | 4819.536 | Trimer |
| 5 | 6426.067 | 6426.048 | Tetramer |

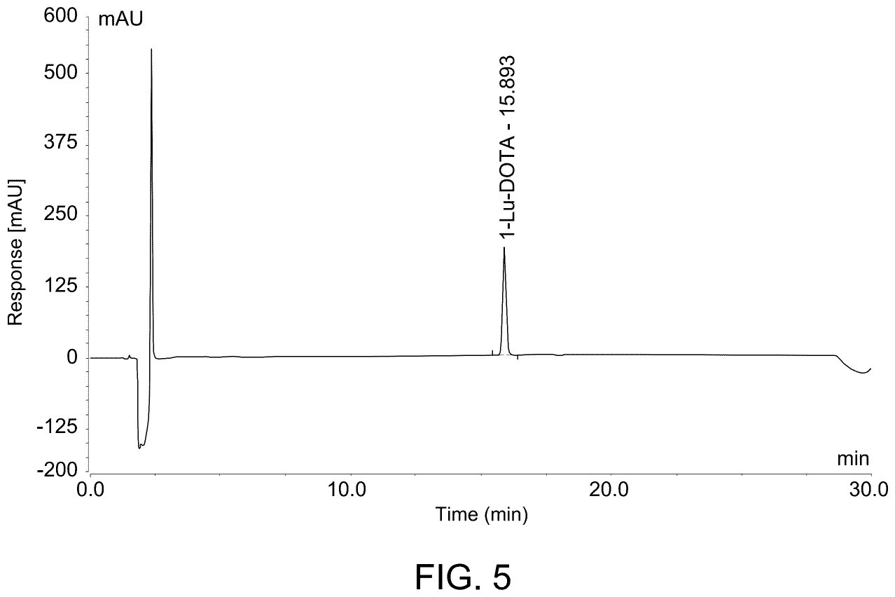

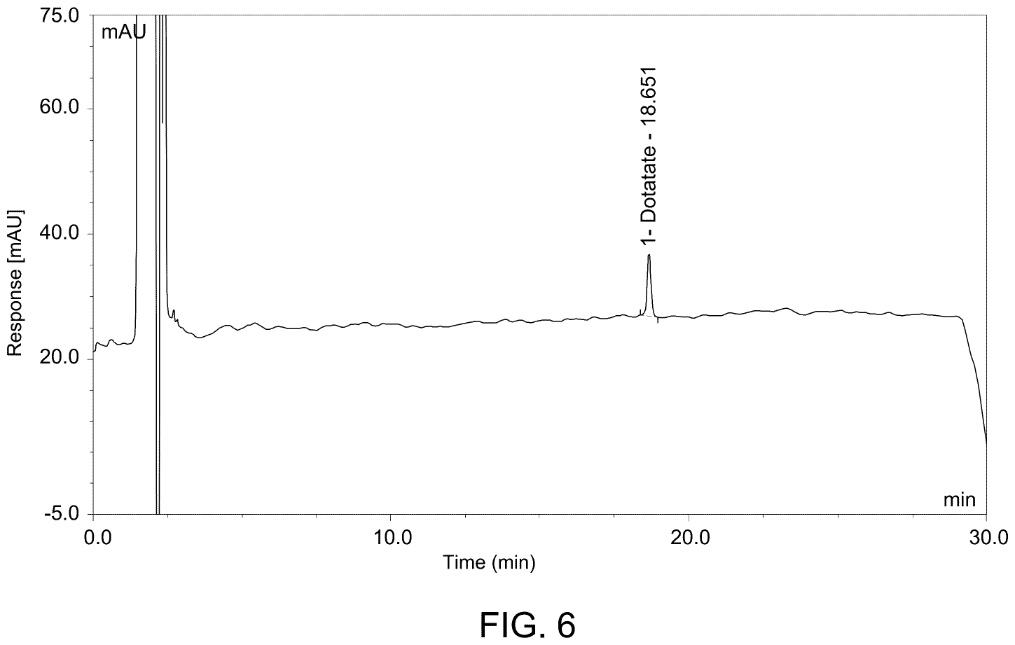

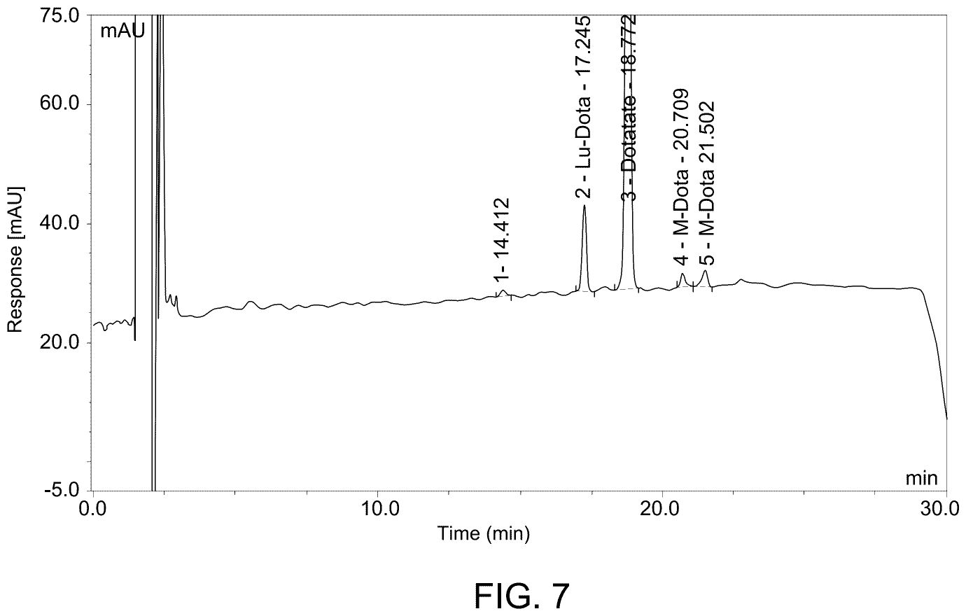

The structure of the ¹⁷⁷Lu-DOTA-TATE drug substance may be further elucidated by comparing both the HPLC chromatograms and the infrared spectra of the Lu-DOTA-TATE and DOTA-TATE reference standards. The HPLC data of the Lu-complex and the DOTA-TATE ligand are provided in FIG. 5 and FIG. 6. The relative retention time (RRT) of the Lu-DOTA-TATE relative to DOTA-TATE is 0.86. This data confirms the identity of the peak in the UV chromatogram depicted in FIG. 7 for the drug product with a retention time of 17.24 minutes as Lu-DOTA-TATE (RRT 0.91) relative to the DOTA-TATE peak at 18.77 minutes. Further characterization may be performed by comparing the IR spectra for Lu-DOTA-TATE to that of the DOTA-TATE ligand by comparing the overlay spectra to determine if there is a distinguishing stretch.

### Stabilizers and Chelator

The ¹⁷⁷Lu-DOTA-TATE drug formulations of the disclosure comprise stabilizers (a stabilizer component) and a chelator.

In any of the various embodiments of the disclosure, at least the stabilizers ethanol and sodium ascorbate are added to the ¹⁷⁷Lu-DOTA-TATE drug formulations to inhibit degradation of the compounds due to radioactivity (also termed radiolysis). Without being bound to any particular theory, it is believed that the stabilizers function as antioxidants or radical scavenger molecules. Non-limiting examples of other such compounds include, for instance, ascorbic acid, gentisic acid, acetic acid, and sodium acetate.

In particular, degradation of compounds due to radioactivity, or radiolysis, is a well-known phenomenon for radiopharmaceuticals. See Baudhuin H, Cousaert J, Vanwolleghem P, et al., Laying the Foundation for an Anti-Radiolytic Formulation for NOTA-sdAb PET Tracers, Pharmaceuticals. 2021; 14(448):1-16, the contents of which are incorporated herein in its entirety. Radionuclides can, to a low extent, induce direct damage due to direct ionization of the radiopharmaceutical by the emitted ionizing radiation. More commonly, ionizing radiation (including 7-radiation) induces damage by the formation of a variety of free radicals such as hydroxyl radicals (HO•), aqueous electron (e_{aq}⁻), superoxide (O₂•⁻) or other highly reactive species (such as H2O⁺, H2O•, and H₂O₂), mainly by interaction with water molecules in aqueous drug formulation solutions. These highly reactive compounds subsequently degrade organic compounds, such as peptides, proteins, DNA-sequences etc. See Baudhuin, et al. Stabilization and protection of a radiopharmaceutical from the effects of radiolytic degradation is important both during long-term storage and at time of radiosynthesis.

In any of the various embodiments of the disclosure, ethanol is added as a stabilizer (anti-oxidant) to the ¹⁷⁷Lu-DOTA-TATE drug formulations. Suitable ethanol concentrations include 1.0 v/v %, 1.1 v/v %, 1.2 v/v %, 1.3 v/v %, 1.4 v/v %, 1.5 v/v %, 1.6 v/v %, 1.7 v/v %, 1.8 v/v %, 1.9 v/v %, 2.0 v/v %, 2.1 v/v %, 2.2 v/v %, 2.3 v/v %, 2.4 v/v %, 2.5 v/v %, 2.6 v/v %, 2.7 v/v %, 2.8 v/v %, 2.9 v/v %, and 3.0 v/v %, and any range constructed therefrom. Non-limiting examples of ranges include from 1.0 v/v % to 3.0 v/v %, from 1.0 v/v % to 2.0 v/v %, from 1.1 v/v % to 1.9 v/v %, from 1.2 v/v % to 1.8 v/v %, from 1.3 v/v % to 1.7 v/v %, from 1.4 v/v % to 1.6 v/v %, from 1.5 v/v % to 1.7 v/v %, from 2.0 v/v % to 3.0 v/v %, from 2.1 v/v % to 2.9 v/v %, from 2.2 v/v % to 2.8 v/v %, from 2.3 v/v % to 2.7 v/v %, and from 2.4 v/v % to 2.6 v/v %.

In any of the various embodiments of the disclosure, sodium ascorbate is added as a stabilizer (anti-oxidant) to the ¹⁷⁷Lu-DOTA-TATE drug formulations. Sodium ascorbate further functions as a buffer. In any of the various embodiments of the disclosure, sodium ascorbate is incorporated into the reaction buffer to act as a radioprotectant against radiolytic breakdown of the non-radioactive precursor and radiolabeled drug substance before it can be diluted into the drug formulation. Additional sodium ascorbate is present in the dilution buffer used for forming the final drug formulation. Suitable sodium ascorbate concentrations in the drug formulation include 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, 30 mg/mL, 31 mg/mL, 32 mg/mL, 33 mg/mL, 34 mg/mL, 35 mg/mL, 36 mg/mL, 37 mg/mL, 38 mg/mL, 39 mg/mL, 40 mg/mL, and any range constructed therefrom. Non-limiting examples of ranges include from 10 mg/mL to 40 mg/mL, from 10 mg/mL to 35 mg/mL, from 10 mg/mL to 30 mg/mL, from 10 mg/mL to 25 mg/mL, from 11 mg/mL to 19 mg/mL, from 12 mg/mL to 18 mg/mL, from 13 mg/mL to 18 mg/mL, from 14 mg/mL to 17 mg/mL, and from 14 mg/mL to 16 mg/mL.

In any of the various embodiments of the disclosure, diethylenetriamine pentaacetic acid (DTPA) excipient is added as a chelator to the ¹⁷⁷Lu-DOTA-TATE drug formulations. DTPA is known to complex metals, is approved for use in injectable drugs, and is included as an excipient in the drug formulation to chelate any possible free ¹⁷⁷Lu, prior to injection. When injected, the chelated radioactive complex formed is rapidly excreted to prevent accumulation of free (non-chelated)¹⁷⁷Lu in the bone. See Breeman W A P, Van der Wansem K, Bernard B F, et al., The addition of DTPA to [177Lu-DOTA0,Tyr3]octreotate prior to administration reduces rat skeleton uptake of radioactivity, Eur J Nucl Med Mol Imaging. 2003; 30(2):312-315, the contents of which are incorporated herein in its entirety. Suitable DTPA concentrations in the ¹⁷⁷Lu-DOTA-TATE drug formulation include 0.04 mg/mL, 0.045 mg/mL, 0.046 mg/mL, 0.047 mg/mL, 0.048 mg/mL, 0.049 mg/mL, 0.05 mg/mL, 0.051 mg/mL, 0.052 mg/mL, 0.053 mg/mL, 0.054 mg/mL, 0.055 mg/mL, and 0.06 mg/mL, and any range constructed therefrom. Non-limiting examples of ranges include from 0.04 mg/mL to 0.06 mg/mL and from 0.045 mg/mL to 0.055 mg/mL.

### ¹⁷⁷Lu-DOTA-TATE Drug Product Synthesis and Drug Formulation Preparation

The following is a non-limiting example of a manufacturing schedule, calibration times, and activity and expiration calculations that are conducted for the drug product. On Day 0, at approximately 20:00 ET, synthesis of the drug product begins. A calculation is made of the drug product radioactivity present on Day 1, at approximately 17:00 ET, hereinafter referred to as the “Drug Product Calibration Time” (approximately 21 hours after drug product formulation is complete). The amount of radioactivity present in the drug product at 17:00 ET on Day 3, is hereinafter referred to as the “Reference Administration Time” (approximately 69 hours after drug product formulation is complete). Day 3 is the day the drug product is scheduled for administration to a patient. The drug product will be identified as expiring at 20:00 ET on Day 3 (approximately 72 hours after drug product formulation is complete).

Radiolabeled ¹⁷⁷Lu-DOTA-TATE is prepared from ¹⁷⁷LuCl₃ and DOTA-TATE in a reaction buffer to form ¹⁷⁷Lu-DOTA-TATE drug substance followed by dilution thereof with a dilution buffer comprising stabilizers and a chelator to form the ¹⁷⁷Lu-DOTA-TATE drug product formulation.

### ¹⁷⁷Lu-DOTA-TATE Radiolabeling

Radiolabeled ¹⁷⁷Lu-DOTA-TATE is prepared in a radiolabeling step comprising the following order of steps (i) to (iii).
(i) in a reactor, form a reaction solution by combining, ¹⁷⁷LuCl₃, DOTA-TATE in solution in a reaction buffer comprising the stabilizer sodium ascorbate, and additional reaction buffer comprising the stabilizer sodium ascorbate.
(ii) preheat the reactor contents to a radiolabeling reaction temperature. In some embodiments, the radiolabeling reaction temperature is about 80° C., is about 85° C., is about 90° C., about 95° C., about 100° C., about 105° C., about 110° C., about 115° C., or about 120° C. and any range constructed therefrom, such as from about 80° C. to about 90° C., from about 90° C. to about 115° C., from about 100° C. to about 115° C., from about 105° C. to about 115° C., from about 105° C. to about 110° C. from about 110° C. to about 115° C., or from about 115° C. to about 120° C.
(iii) react the reactor contents at the radiolabeling reaction temperature for a reaction time to form a solution of drug product ¹⁷⁷Lu-DOTA-TATE. In some embodiments, the radiolabeling reaction time is about 5 minutes, about 10 minutes, about 15 minutes, about 20 minutes, about 25 minutes, about 30 minutes, about 35 minutes, about 40 minutes, or about 45 minutes and any range constructed therefrom, such as from about 5 minutes to about 10 minutes, from about 10 minutes to about 20 minutes, from about 20 minutes to about 25 minutes, from about 25 minutes to about 30 minutes, from about 30 minutes to about 35 minutes, from about 35 minutes to about 40 minutes, from about 40 minutes to about 45 minutes, from about 5 minutes to about 15 minutes, from about 5 minutes to about 20 minutes, from about 5 minutes to about 25 minutes, from about 5 minutes to about 30 minutes, from about 5 minutes to about 35 minutes, from about 5 minutes to about 40 minutes, or from about 5 minutes to about 45 minutes,

The reaction buffer is used to dissolve the DOTA-TATE raw material and as a rinse after the ¹⁷⁷Lu and DOTA-TATE buffer solution are added to the reactor. In one non-limiting embodiment, the reaction buffer may be prepared by adding 4.20 g (±2%, 4.11 to 4.28 g) of sodium ascorbate to a 100 mL volumetric flask containing approximately 50 mL of water for injection (WFI). 240 μL of concentrated HCl is then added to the flask and the flask is brought to the mark using WFI. The pH of the reaction buffer is checked using pH paper, with an acceptance range of 4.7 to 5.3. If the pH of the dilution buffer is greater than 5.3, the pH may be optionally adjusted with an acid, such as hydrochloric acid. The acceptable range of sodium ascorbate concentration in the reaction buffer is about 5 mg/mL, about 10 mg/mL, about 20 mg/mL, about 30 mg/mL, about 40 mg/mL, about 50 mg/mL, about 60 mg/mL, about 70 mg/mL, or about 80 mg/mL, and any range constructed therefrom, such as from about 5 mg/mL to about 80 mg/mL, from about 10 mg/mL to about 70 mg/mL, from about 20 mg/mL to about 60 mg/mL, or from about 30 mg/mL to about 50 mg/mL.

In some embodiments, the DOTA-TATE buffer solution suitably has a DOTA-TATE concentration of about 0.5 mg/mL, about 1 mg/mL, about 1.5 mg/mL, about 5 mg/mL, about 10 mg/ml, or about 20 mg/mL, and any range constructed therefrom, such as from about 0.5 mg/mL to about 20 mg/mL, from about 0.5 mg/mL to about 10 mg/mL, from about 0.5 mg/mL to about 5 mg/mL, or from about 0.5 mg/mL to about 1.5 mg/mL. In some embodiments, the DOTA-TATE buffer solution is prepared by adding reaction buffer to the DOTA-TATE raw material, such as contained in a vial, to achieve a desired concentration, such as for instance, about 1 mg/mL. In some embodiments, dissolved DOTA-TATE buffer solution is transeptally removed from each container, such as a vial, and added to a tared container (such as a Trasis peptide vial). In some embodiments, the container is a vial and the target net weight of the DOTA-TATE buffer solution, considering minor losses to the individual vials, is 6.9 g, with an acceptance range of 6.5-7.2 g.

The ratio of reaction buffer to DOTA-TATE buffer can be described in terms of radiolabeling batch size at Drug Product Calibration Time. In some embodiments, the batch size at Drug Product Calibration Time is about 250 mCi, about 500 mCi, about 1000 mCi, about 2000 mCi, about 3000 mCi, about 4000 mCi, about 5000 mCi, about 6000 mCi, about 7000 mCi, about 8000 mCi, about 9000 mCi, about 10000 mCi, about 11000 mCi, about 12000 mCi, about 13000 mCi, about 14000 mCi, and about 15000 mCi, and any range constructed therefrom, such as from about 250 mCi to about 15000 mCi, from about 1000 mCi to about 14000 mCi, from about 2000 mCi to about 13000 mCi, from about 3000 mCi to about 12000 mCi, from about 4000 mCi to about 10000 mCi, from about 5000 to about 9000, from about 6000 to about 8000, or from about 3000 mCi to about 10000 mCi. In some embodiments the volume ratio of reaction buffer to DOTA-TATE buffer is suitably about 3:1, about 2.5:1, 2:1, about 1.5:1, about 1:1, about 1:1.5, about 1:2, about 1:2.5 or about 1:3 at Drug Product Calibration Time. In some embodiments, the ratio is about 2:1 per 1000 mCi of activity. In some embodiments, the ratio is from about 1:15 to about 1:2.5 per 3000 mCi to 5200 mCi of activity at Drug Product Calibration Time. The combination of reaction buffer and DOTA-TATE raw material dissolved in reaction buffer results in a sodium ascorbate concentration during radiolabeling as described elsewhere herein. In some embodiments, the sodium ascorbate concentration is from about 25 mg/mL to about 31 mg/mL or from about 30 to about 31 mg/mL. The sodium ascorbate concentration range provides for ¹⁷⁷Lu-DOTA-TATE with an RCP of greater than 95%.

Non-limiting examples, based on experimentation to date, of sodium ascorbate concentration in ¹⁷⁷Lu-DOTA-TATE batches are provided in Table E below where: ¹⁷⁷Lu is in 0.05 M HCl at a concentration of 2 Ci/mL at Drug Product Calibration Time; “NaAscb” refers to sodium ascorbate; the reaction buffer and DOTA-TATE buffer each comprise 42 mg/mL NaAscb; “total volume” refers to the total of the reaction buffer and the DOTA-TATE buffer; and “total reaction volume” refers to the total of the reaction buffer, the DOTA-TATE buffer, and the ¹⁷⁷Lu. Further in Table E below, “x” refers to the possibility of scale-up to 10 Ci and 15 Ci batch size where multiple reactions at 5 Ci can be combined into a single formulation, such as for instance where “2×3.0” and “3×7.2” refer to two additions of 3.0 mL and three additions of 7.2 mL, respectively.

TABLE E

| | 3000 | 4000 | 5000 | 10000 | 15000 |
|---|---|---|---|---|---|
| Batch size | mCi | mCi | mCi | mCi | mCi |
| ¹⁷⁷Lu volume (mL) | 1.8 | 2.5 | 3.0 | 2 × 3.0 | 3 × 3.0 |
| Reaction buffer | 2.0 | 2.0 | 2.0 | 2 × 2.0 | 3 × 2.0 |
| volume (mL) | | | | | |
| DOTA-TATE buffer | 3.0 | 4.4 | 5.2 | 2 × 5.2 | 3 × 5.2 |
| volume (mL) | | | | | |
| Total volume (mL) | 5.0 | 6.4 | 7.2 | 2 × 7.2 | 3 × 7.2 |
| Total NaAscb (mg) | 210.0 | 268.8 | 302.4 | 604.8 | 907.2 |
| Total Reaction | 6.8 | 8.9 | 10.2 | 2 × 10.2 | 3 × 10.2 |
| volume (mL) | | | | | |
| Reaction NaAscb | 30.9 | 30.2 | 29.9 | 29.9 | 29.9 |
| concentration (mg/mL) | | | | | |

For the reaction mixture, in some embodiments, the concentration range of ¹⁷⁷LuCl₃ in the radiolabeling reaction is suitably about 20 mCi/mL, about 30 mCi/mL, about 40 mCi/mL, about 50 mCi/mL, about 60 mCi/mL, about 70 mCi/mL, about 80 mCi/mL, about 90 mCi/mL, about 100 mCi/mL, about 110 mCi/mL, about 120 mCi/mL, or about 130 mCi/mL, and any range constructed therefrom, for total radioactivity at time of labeling of from about 126 to about 346 mCi. In some embodiments, the concentration of ¹⁷⁷LuCl₃ in the radiolabeling reaction is suitably about 100 mCi/mL, about 110 mCi/mL, about 120 mCi/mL, about 130 mCi/mL, about 140 mCi/mL, about 150 mCi/mL, about 160 mCi/mL, or about 170 mCi/mL, and any range constructed therefrom, for total radioactivity at time of labeling of from about 615 mCi to about 1027 mCi. In some embodiments, the concentration of ¹⁷⁷LuCl₃ in the radiolabeling reaction is suitably about 400 mCi/mL, 500 mCi/mL, 600 mCi/mL, 700 mCi/mL, 800 mCi/mL, 900 mCi/mL, or 950 mCi/mL, and any range constructed therefrom, for total radioactivity at time of labeling of from about 3811 mCi to about 9107 mCi. In any of the various embodiments, the radiolabeling reaction has an RCP of greater than 95%.

In some embodiments, DOTA-TATE buffer is prepared at a target concentration of about 0.3 mg/mL, 0.4 mg/mL, about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL, about 1.5 mg/mL, about 1.6 mg/mL, about 1.7 mg/mL, about 1.8 mg/mL, about 1.9 mg/mL, or about 2.0 mg/mL, and any range constructed therefrom, such as from about 0.3 mg/mL to about 2 mg/mL, from about 0.3 mg/mL to about 1.6 mg/mL, or from about 0.8 mg/mL to about 1.2 mg/mL. In any of the various embodiments, the concentration produces drug product with an RCP of greater than 95%. In other embodiments, the mole ratio of DOTA-TATE to ¹⁷⁷Lu is about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, and any range constructed therefrom, such as from about 5:1 to about 20:1, from about 7:1 to about 15:1, or from about 10:1 to about 13:1. In some embodiments, the target amount of DOTA-TATE in the radiolabeling reaction is 1 mg/Ci of ¹⁷⁷Lu activity at Drug Product Calibration Time. Examples of some molar ratios, based on experimentation to date, are shown in Table F where “Radio Activity” refers to Radiolabeling Activity.

TABLE F

| | DOTA-TATE:¹⁷⁷Lu | Radioactivity | RCP |
|---|---|---|---|
| Example | mol ratio | (mCi) | (%) |
| 1 | 19 | 144 | 97.9 |
| 2 | 8.9 | 615 | 98.3 |
| 3 | 7.9 | 123 | 98.3 |
| 4 | 7.3 | 188 | 97.4 |
| 5 | 6.7 | 204 | 97.3 |
| 6 | 6.6 | 207 | 97.1 |
| 7 | 6.5 | 209 | 97.6 |
| 8 | 6.3 | 124 | 98.3 |
| 9 | 5.4 | 180 | 97.8 |
| 10 | 5.3 | 1027 | 97.5 |
| 11 | 5.3 | 183 | 97.6 |
| 12 | 3.9 | 125 | 97.1 |
| 13 | 2.0 | 124 | 67.7 |

The ¹⁷⁷Lu-DOTA-TATE radiolabeling step is suitably done at a pH of about 4, about 4.5, about 5, about 5.5, or about 6, and any range constructed therefrom, such as from about 4 to about 6.5, from about 4.5 to about 6, from about 4.5 to about 5.5, from about 4.5 to about 5.0, or from about 4.9 to about 5.1.

The ¹⁷⁷Lu-DOTA-TATE radiolabeling step reaction time is not narrowly limited, and in some embodiments is suitably about 5 minutes, about 10 minutes, about 15 minutes, about 20 minutes, about 25 minutes, about 30 minutes, about 35 minutes, about 40 minutes, or about 45 minutes, and any range constructed therefrom, such as from about 5 minutes to about 45 minutes, from about 10 minutes to about 40 minutes, from about 15 minutes to about 35 minutes, or from about 20 minutes to about 30 minutes.

### ¹⁷⁷Lu-DOTA-TATE Drug Formulation Preparation

¹⁷⁷Lu-DOTA-TATE drug formulation is prepared by (i) diluting radiolabeled ¹⁷⁷Lu-DOTA-TATE with a dilution buffer comprising stabilizers and a chelator to form a bulk ¹⁷⁷Lu-DOTA-TATE drug product at the correct radioactivity concentration followed by (ii) filtration thereof through a sterile filter and dispensing of the ¹⁷⁷Lu-DOTA-TATE drug product.

In some embodiments of the disclosure, the solution of ¹⁷⁷Lu-DOTA-TATE drug substance from the reactor is directly combined with the dilution buffer in the absence of any intervening process steps. Alternatively stated, in such embodiments, the radiolabeled ¹⁷⁷Lu-DOTA-TATE is not isolated prior to combination with the dilution buffer. In some embodiments, after preparation of the ¹⁷⁷Lu DOTA-TATE drug substance in the radiolabeling reaction, the reactor contents are transferred directly to a formulation container (e.g., a vial or bottle) that has been filled with a pre-determined amount of dilution buffer, and is therefore not isolated during the process. The radioactivity concentration in the reactor is sufficiently high to promote complete incorporation of the ¹⁷⁷Lu⁺³; however, an extended amount of time in the concentrated form can lead to radiolysis of the drug substance. In some embodiments, transfer of the radiolabeling reaction can be performed manually. In some embodiments, transfer of the radiolabeling reaction can be performed using automated equipment. The combination of automation and direct transfer to the formulation container reduces risk of radiolysis and process upset, and provides for process advantages of avoiding the isolation step known in the prior art and the associated equipment and safety and environmental hazards associated with the radioisotope.

The volume of dilution buffer may suitably be about 0.3 mL/Ci, about 0.4 mL/Ci, about 0.5 mL/Ci, about 0.6 mL/Ci, about 0.7 mL/Ci, about 0.8 mL/Ci, about 0.9 mL/Ci, about 1.0 mL/Ci, about 1.1 mL/Ci, about 1.2 mL/Ci, about 1.3 mL/Ci, about 1.4 mL/Ci, about 1.5 mL/Ci, or about 1.6 mL/Ci, and any range constructed therefrom, such as for instance from about 0.3 mL/Ci to about 1.6 mL/Ci or from about 0.8 mL/Ci to about 1.2 mL/Ci.

A suitable batch size is not narrowly limited. In some non-limiting embodiments, the batch size at Drug Product Calibration Time is suitably about 250 mCi, about 500 mCi, about 1000 mCi, about 2000 mCi, about 3000 mCi, about 4000 mCi, about 5000 mCi, about 6000 mCi, about 7000 mCi, about 8000 mCi, about 9000 mCi, about 10,000 mCi, about 11000 mCi, about 12000 mCi, about 13000 mCi, about 14000 mCi, or about 15000 mCi and any range constructed therefrom, such as from about 250 mCi to about 10,000 mCi, or from about 3000 mCi to about 5000 mCi.

The concentration of the stabilizers and chelator in the dilution buffer and the ratio of dilution buffer to the solution of radiolabeled ¹⁷⁷Lu-DOTA-TATE are selected to provide for a ¹⁷⁷Lu-DOTA-TATE drug formulation having the characteristics defined herein.

For instance, the ¹⁷⁷Lu-DOTA-TATE drug formulation may comprise a therapeutically effective amount of ¹⁷⁷Lu-DOTA-TATE of from 222 mCi to 272 mCi, from 230 mCi to 265 mCi, or from 230 mCi to 260 mCi, at Drug Product Calibration Time, which is typically 48-hours before Reference Administration Time.

The ¹⁷⁷Lu-DOTA-TATE drug formulation may comprise an ethanol concentration of about 1.0 v/v %, about 1.5 v/v %, about 2.0 v/v %, about 2.5 v/v %, or about 3.0 v/v %, and any range constructed therefrom, such as from 1.0 v/v % to 2.0 v/v %, from 1.1 v/v % to 1.9 v/v %, from 1.2 v/v % to 1.8 v/v %, from 1.3 v/v % to 1.7 v/v %, from 1.4 v/v % to 1.6 v/v %, from 1.5 v/v % to 1.7 v/v %, from 2.0 v/v % to 3.0 v/v %, from 2.1 v/v % to 2.9 v/v %, from 2.2 v/v % to 2.8 v/v %, from 2.3 v/v % to 2.7 v/v %, or from 2.4 v/v % to 2.6 v/v %. In some embodiments, the ethanol concentration is about 1.5 v/v %. In some embodiments, the ethanol concentration is about 2.5 v/v %.

The ¹⁷⁷Lu-DOTA-TATE drug formulation may comprise a sodium ascorbate concentration of about 7 mg/mL, about 10 mg/mL, about 15 mg/mL, about 20 mg/mL, about 25 mg/mL, about 30 mg/mL, about 35 mg/mL, or about 40 mg/mL, and any range constructed therefrom, such as from about 7 mg/mL to about 40 mg/mL, from about 10 mg/mL to about 30 mg/mL, from about 10 mg/mL to about 25 mg/mL, from about 10 mg/mL to about 20 mg/mL, from about 11 mg/mL to about 19 mg/mL, from about 12 mg/mL to about 18 mg/mL, from about 13 mg/mL to about 18 mg/mL, from about 14 mg/mL to about 17 mg/mL, or from about 14 mg/mL to about 16 mg/mL. In some embodiments, the sodium ascorbate concentration is about 15 g/mL.

The ¹⁷⁷Lu-DOTA-TATE drug formulation may comprise a DTPA concentration of from about 0.04 mg/mL to about 0.06 mg/mL.

Filtration may suitably be done by sterile filtration methods known in the art. For instance, suitable filters may have a pore size of about 0.2 μm. One non-limiting example of a filter is a commercially available from Pall, having a 0.2 μm Supor™ (polyethersulphone) membrane. Single and dual filter systems are within the scope of the present disclosure.

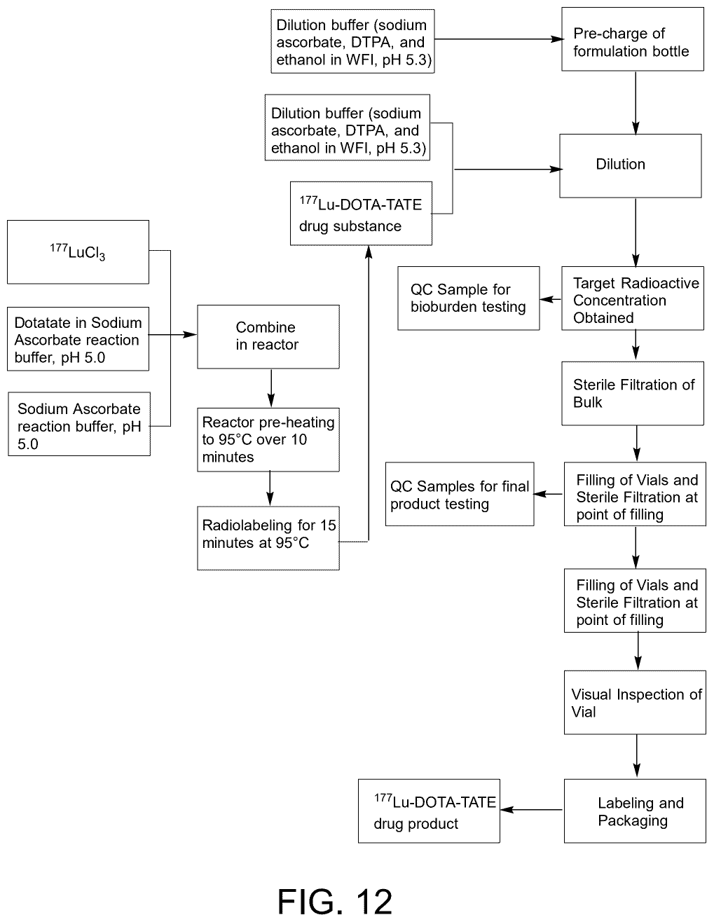

In some embodiments of the disclosure, ¹⁷⁷Lu-DOTA-TATE drug formulation is prepared according to the process flow diagram depicted in FIG. 12.

### ¹⁷⁷Lu-DOTA-TATE Drug Formulation Specifications

¹⁷⁷Lu-DOTA-TATE drug formulations of the disclosure may be characterized by the following specifications of chemical identity, radiochemical identity, DOTA-TATE and related substances assay, radiochemical purity, pH, radioactive concentration, apparent molar activity, total vial assay, unbound ¹⁷⁷Lu as follows, where examples of suitable analytical methods are provided elsewhere herein. All stated specification ranges in paragraph 0111 to 0119 are at Drug Product Calibration Time, which is defined as 48-hours before Reference Administration Time.

Chemical identity. The chemical identity of DOTA-TATE in the drug product as determined by HPLC relative retention time (RRT) of the drug product sample injection is compared to DOTA-TATE in a standard solution of 0.85, 0.86, 0.87, 0.88, 0.89, 0.90, 0.91, 0.92, 0.93, 0.94, 0.95, 0.96, 0.97, 0.98, 0.99, or 1.0, and any range constructed therefrom is within the scope of the disclosure. Suitable such ranges include from 0.85 to 1.0, from 0.90 to 1.0, from 0.91 to 0.99, from 0.92 to 0.98, from 0.93 to 0.97, from 0.93 to 0.96; and from 0.93 to 0.95.

Radiochemical identity. The radiochemical identity of ¹⁷⁷Lu-DOTA-TATE in the drug product as determined by HPLC relative retention time (RRT) of the drug product compared to DOTA-TATE in a standard solution of 0.85, 0.86, 0.87, 0.88, 0.89, 0.90, 0.91, 0.92, 0.93, 0.94, 0.95, 0.96, 0.97, 0.98, 0.99, or 1.0, and any range constructed therefrom is within the scope of the disclosure. Suitable such ranges include from 0.85 to 1.0, from 0.90 to 1.0, from 0.91 to 0.99, from 0.92 to 0.98, from 0.93 to 0.97, and from 0.93 to 0.96.

DOTA-TATE and related substances assay. A DOTA-TATE and related substances assay as measured by HPLC is 8.0 μg/mL, 8.5 μg/mL, 9.0 μg/mL, 9.5 μg/mL, 10.0 μg/mL, 10.5 μg/mL, 11.0 μg/mL, 11.5 μg/mL, or 12.0 μg/mL, and any range constructed therefrom, is within the scope of the disclosure. Suitable such ranges include from 8.0 μg/mL to 12.0 μg/mL, from 8.5 μg/mL to 11.5 μg/mL, from 9.0 μg/mL to 11.0 μg/mL, and from 9.5 μg/mL to 10.5 μg/mL.

Radiochemical purity assay. The ¹⁷⁷Lu-DOTA-TATE drug formulations of the disclosure are characterized by a radiochemical purity assay of not less than 95%, not less than 95.5%, not less than 96%, not less than 96.5%, not less than 97%, not less than 97.5%, not less than 98%, not less than 98.5%, not less than 99%, and not less than 99.5% 72 hours after formulation thereof, up to 4 days after formulation thereof, up to 5 days after formulation thereof, up to 6 days after formulation thereof, or up to 7 days after formulation thereof.

pH. A pH of 4.5, 4.6, 4.7, 4.8, 4.9 5, 5.1, 5.2, 5.3, 5.4, 5.5, 5.6, 5.7, 5.8, 5.9, and 6, and any range constructed therefrom, is within the scope of the disclosure. Suitable ranges include from 4.5 to 6, from 4.8 to 5.5, or from 5 to 5.5.

Radioactive concentration. A radioactive concentration of 9 mCi/mL, 9.1 mCi/mL, 9.2 mCi/mL, 9.3 mCi/mL, 9.4 mCi/mL, 9.5 mCi/mL, 9.6 mCi/mL, 9.7 mCi/mL, 9.8 mCi/mL, 9.9 mCi/mL, 10 mCi/mL, 10.1 mCi/mL, 10.2 mCi/mL, 10.3 mCi/mL, 10.4 mCi/mL, 10.5 mCi/mL, 10.6 mCi/mL, 10.7 mCi/mL, 10.8 mCi/mL, 10.9 mCi/mL, and 11 mCi/mL, and any range constructed therefrom, is within the scope of the disclosure. Suitable ranges include from 9 mCi/mL to 11 mCi/mL, and from 9.5 mCi/mL to 10.5 mCi/mL.

Apparent molar activity. An apparent molar activity of 1.1 mCi/nmol, 1.2 mCi/nmol, 1.3 mCi/nmol, 1.4 mCi/nmol, 1.5 mCi/nmol, 1.6 mCi/nmol, 1.7 mCi/nmol, 1.8 mCi/nmol, 1.9 mCi/nmol, and 2 mCi/nmol, and any range constructed therefrom, is within the scope of the disclosure. Suitable ranges include from 1.1 mCi/nmol to 2 mCi/nmol, from 1.1 mCi/nmol to 1.8 mCi/nmol, and from 1.2 mCi/nmol to 1.6 mCi/nmol.

In embodiments where the container is a vial, a fill volume for a vial contains from 20.5 mL to about 21.0 mL to about 22.0 mL, to about 23.0 mL, to about 24.0 mL to about 25 mL, and any range constructed therefrom, of the ¹⁷⁷Lu-DOTA-TATE drug formulation of the disclosure wherein the total volume delivers a therapeutically effective amount of ¹⁷⁷Lu-DOTA-TATE drug formulation when the contents are administered to a patient in need of ¹⁷⁷Lu-DOTA-TATE therapy. The therapeutically effective amount is 222 mCi/vial, 225 mCi/vial, 230 mCi/vial, 235 mCi/vial, 240 mCi/vial, 245 mCi/vial, 250 mCi/vial, 255 mCi/vial, 260 mCi/vial, 265 mCi/vial, 270 mCi/vial, and 272 mCi/vial, and any range constructed therefrom, is within the scope of the disclosure. Suitable ranges include from 222 mCi/vial to 272 mCi/vial, from 230 mCi/vial to 265 mCi/vial, or from 230 mCi/vial to 260 mCi/vial at Drug Product Calibration Time.

Unbound ¹⁷⁷Lu. An unbound ¹⁷⁷Lu concentration of not more than 1%, not more than 0.9%, not more than 0.8%, not more than 0.7%, not more than 0.6%, not more than 0.5%, not more than 0.4%, not more than 0.3%, not more than 0.2%, and not more than 0.1% is within the scope of the disclosure.

### Example ¹⁷⁷Lu-DOTA-TATE Compositions

Non-limiting examples of ¹⁷⁷Lu-DOTA-TATE drug formulation compositions within the scope of the disclosure are provided below.

One example ¹⁷⁷Lu-DOTA-TATE drug formulation contains 2-3 v/v % ethanol and is characterized as follows in Table G where “vial” refers to a vial containing from 23.0 to 25.0 mL of the ¹⁷⁷Lu-DOTA-TATE drug formulation.

### Table G ¹⁷⁷Lu-DOTA-TATE Composition

| Component | Content |
|---|---|
| Chemical identity | Relative retention time of 0.85 to 1.00 |
| (DOTA-TATE) | compared to DOTA-TATE in HPLC standard |
| Radiochemical identity | Relative retention time of 0.85 to 1.05 |
| (¹⁷⁷Lu-DOTA-TATE) | compared to DOTA-TATE in HPLC standard |
| DOTA-TATE and related | 8.0 to 12.0 μg/mL |
| substance assay | |
| Radiochemical Purity | Not less than 95% |
| Unbound ¹⁷⁷Lu | Not more than 1% |
| Radionuclide identity | Gamma emission spectra at 113 ± 1 keV and |
| | 208 ± 1 keV |
| Radionuclide purity | ¹⁷⁷Lu of not less than 99.9% |
| | ¹⁷⁵Yb of not more than 0.1% |
| | ^{177m}Lu of not more than 0.07% |
| | Total other impurities of not more than 0.01% |
| Sodium Ascorbate | 13 to 17 mg/mL |
| Dehydrated Ethanol | 2.0 to 3.0 v/v % |
| DTPA | 0.04 to 0.06 mg/mL |
| pH | 4.5 to 6.0 |
| Radioactive | 9 to 11 mCi/mL |
| concentration | |
| Apparent molar | 1.1 to 2.0 mCi/nmol at Drug Product |
| activity | Calibration Time |
| Total vial assay | 222 to 272 mCi/vial at Drug Product |
| | Calibration Time |

One example ¹⁷⁷Lu-DOTA-TATE drug formulation contains 1.5 v/v % ethanol and is characterized as follows in Table H.

TABLE H

| | | |
|---|---|---|
| Component | Per mL | Per 25 mL dose |

| | | |
|---|---|---|
| ¹⁷⁷Lu | 0.00008 mg | 0.002 mg |
| | (about 8 mCi) | (about 200 mCi) |

| | | | | |
|---|---|---|---|---|
| DOTA-TATE | 10 | μg | 250 | μg |
| Ethanol^{a} | 11.84 | mg | 296 | mg |
| Sodium Ascorbate | 15 | mg^{b} | 375 | mg^{b} |
| DTPA | 0.05 | mg | 1.25 | mg |

| | | |
|---|---|---|
| Water | q.s. | q.s. |
| NaOH/HCl | As needed for | As needed for |
| | pH adjust. | pH adjust. |

^{a}Based on a density of 0.789 g/mL
^{b}Mass is as mg ascorbate

One example ¹⁷⁷Lu-DOTA-TATE drug formulation contains 2.5 v/v % ethanol and is characterized as follows in Table I.

TABLE I

| | | |
|---|---|---|
| Component | Per mL | Per 25 mL dose |

| | | |
|---|---|---|
| ¹⁷⁷Lu | 0.00008 mg | 0.002 mg |
| | (about 8 mCi) | (about 200 mCi) |

| | | | | |
|---|---|---|---|---|
| DOTA-TATE | 10 | μg | 250 | μg |
| Ethanol^{a} | 19.72 | mg | 493 | mg |
| Sodium Ascorbate | 15 | mg^{b} | 375 | mg^{b} |
| DTPA | 0.05 | mg | 1.25 | mg |

| | | |
|---|---|---|
| Water | q.s. | q.s. |
| NaOH/HCl | As needed for | As needed for |
| | pH adjust. | pH adjust. |

^{a}Based on a density of 0.789 g/mL
^{b}Mass is as mg ascorbate

In Table J below, a prior art ¹⁷⁷Lu-DOTA-TATE composition is compared to ¹⁷⁷Lu-DOTA-TATE compositions of the disclosure comprising 1.5 v/v % and 2.5 v/v % ethanol, respectively. The prior art composition was formulated to provide a composition representative of the LUTHATERA® FDA listed drug formulation package insert. The mass of ¹⁷⁷Lu-DOTA-TATE is based on the nominal radioactivity per dose and a specific activity of 95.0 mCi/μg. DOTA-TATE is added in excess in the drug substance labeling process to achieve the same molar activity of ligand as the formulated reference formulation. NaOH and HCl are added to the compositions of the disclosure as needed to adjust pH.

TABLE J

| | | Composition (mg/dose) | | | | | |
|---|---|---|---|---|---|---|---|
| Component | Function | Prior art | | 1.5 v/v % EtOH | | 2.5 v/v % EtOH | |
| ¹⁷⁷Lu-DOTA-TATE | Drug/API | 0.02 | mg | 0.02 | mg | 0.02 | mg |
| DOTA-TATE | Ligand | 0.25 | mg | 0.250 | mg | 0.250 | mg |
| DTPA | Chelator | 1.0-1.3 | mg | 1.25 | mg | 1.25 | mg |

| | | | | | |
|---|---|---|---|---|---|
| Ascorbic Acid | Buffer/Antiox. | 56-70 | mg | None | None |

| | | | | | | |
|---|---|---|---|---|---|---|
| Na Ascorbate | Buffer/Antiox. | None | 375 | mg | 375 | mg |
| Ethanol | Antioxidant | None | 296 | mg | 493 | mg |

| | | | | | |
|---|---|---|---|---|---|
| Acetic Acid | Buffer | 9.6-12 | mg | None | None |
| Na Acetate | Buffer | 13.2-16.5 | mg | None | None |
| Gentisic Acid | Antioxidant | 12.6-15.8 | mg | None | None |
| NaOH | pH Adjuster | 13-16.3 | mg | As needed | As needed |
| NaCl | Tonicity Agent | 137-171.5 | mg | None | None |

| | | | | |
|---|---|---|---|---|
| HCl | pH Adjuster | None | As needed | As needed |
| Water | Diluent | q.s. | q.s. | q.s. |

Further comparison of a prior art ¹⁷⁷Lu-DOTA-TATE reference composition to a ¹⁷⁷Lu-DOTA-TATE composition of the disclosure containing 2.5 v/v % ethanol composition is reproduced below in Table K with components, except for ¹⁷⁷Lu, on a mg/mL basis.

TABLE K

| | Composition (mg/mL) | | |
|---|---|---|---|
| Component | | Prior art | 2.5 v/v % EtOH |

| | | | | |
|---|---|---|---|---|
| DOTA-TATE | 10 | μg | 10 | μg |
| DTPA | 0.05 | mg | 0.05 | mg |
| Na Ascorbate | 2.8 | mg | 15.0 | mg |

| | | | |
|---|---|---|---|
| Ethanol | None | 19.7 | mg |

| | | | |
|---|---|---|---|
| Acetic Acid | 0.48 | mg | None |
| Na Acetate | 0.66 | mg | None |
| Gentisic Acid | 0.63 | mg | None |
| NaOH | 0.65 | mg | As needed |
| NaCl | 6.85 | mg | None |

| | | |
|---|---|---|
| HCl | None | As needed |
| Water | q.s. | q.s. |

### Methods of Administering the ¹⁷⁷Lu-DOTA-TATE Drug Formulation

Administration of the drug formulation to a patient comprises the steps of: (a) calibrating dose activity of a ¹⁷⁷Lu-DOTA-TATE injection at a Reference Administration Time; (b) using the ¹⁷⁷Lu-DOTA-TATE injection within about 3 hours after Reference Administration Time; (c) using aseptic technique and radiation shielding when withdrawing and administering ¹⁷⁷Lu-DOTA-TATE injection; (d) inspecting the ¹⁷⁷Lu-DOTA-TATE injection visually for particulate matter and discoloration before administration and only using it if the solution does not contain particulate matter or is discolored; (e) calculating the necessary volume to administer based on measured activity, volume, Reference Administration Time, and date; (f) using a dose calibrator to measure the patient dose immediately prior to administration of the drug formulation; (g) after injection of the ¹⁷⁷Lu-DOTA-TATE injection, an intravenous flush of 0.9% sodium chloride injection, USP is administered to the patient; and (h) any unused drug is disposed in a safe manner in compliance with applicable regulations.

### Analytical Methods

### HPLC Method

An HPLC method may be used to determine the radiochemical purity (RCP) and quantity of ¹⁷⁷Lu-DOTA-TATE and related substances in a sample of the finished drug product.

In one HPLC method, an Agilent 1260 Infinity II HPLC column and LabLogic RAD detector may suitably be used with Mobile Phase A of 0.1% trifluoracetic acid (TFA) in water and Mobile Phase B of 0.1% TFA in acetonitrile.

In such a method, the LC column in the HPLC is flushed with mobile phase at 1.0 mL/min for approximately 30 minutes with an 85:15 ratio of Mobile Phase A (0.1% Trifluoracetic Acid (TFA) in Water): Mobile Phase B (0.1% Trifluoracetic Acid (TFA) in Acetonitrile). The UV detector and radiometric detector are activated and allowed to equilibrate. Gradient and HPLC conditions are shown in the following two tables.

Gradient

| Time | % Mobile | % Mobile |
|---|---|---|
| (min) | Phase A | Phase A |
| 0 | 85 | 15 |
| 20 | 75 | 25 |
| 26 | 75 | 25 |
| 27 | 85 | 15 |
| 30 | 85 | 15 |

HPLC Conditions

| | | |
|---|---|---|
| Flow Rate | 1.0 | mL/min |
| Column Temperature | 35° | C. |
| Injection Volume, RCP | 10 | μL |
| Injection Volume, Quantitative: | 100 | μL |
| Diluent, Standards, and Sample | | |
| Run Time | 30 | min |
| UV Wavelength | 220 | nm |

| | |
|---|---|
| Radioactivity Detector Setting | Adjust to maximize use |
| | of detector's full scale |

### Radionuclidic Purity (RNP) Measurement, Radionuclide Identity (RNID) Measurement, Radiochemical Impurities Measurement, and LOQ Measurement

RNP, RNID, radiochemical impurities, and LOQ may be measured according to the following method.

As used herein, critical limit or limit of detection(“LOD”) refers to the smallest amount of activity of an analyte in the sample (in the presence of a sample) that will be detected with a probability (i.e., 95% confidence) of non-detection (Type II error) while accepting a probability of erroneously deciding that a positive quantity of analyte is present (Type I error).

As used herein, Minimum Detectable Activity (MDA) (“LOQ”) refers to the smallest amount of activity of an analyte in the system (no sample present) that will be detected with a probability (i.e., 95% confidence) of non-detection (Type II error) while accepting a probability of erroneously deciding that a positive quantity of analyte is present (Type I error). (Gilmore, G. 2008. Practical Gamma-ray Spectrometry. 2^{nd} Ed. Wiley. 119-120.)

As used herein, Dead Time refers to the time in which the signal entering the detector is being processed, thereby preventing the processing of additional signal during the time interval. This is a metric used to determine if a sample can be properly analyzed without additional interferences (i.e., increased summing events).

Initial Dilution. Obtain a labeled 10 mL glass tubing vial and add 4.5 mL of diluent to the vial. Transfer 0.5 mL of QC sample to the vial. Alternatively, use the results from RAC and proceed to final sample preparation. Mix the contents thoroughly and obtain a dose calibration reading. Using the results from above, calculate the concentration of the dilution.

Final sample preparation for HPGe detector. Calculate the amount of sample needed from dilution 1 to prepare a 75 μCi sample. Then round to the nearest 5 μL due to the volume increment of the 5 mL pipette. Using a pipette, add the calculated amount to a labelled 7 mL scintillation vial. Using a pipette, bring the assay vial to a final volume of 2.0 mL with diluent. Mix thoroughly without inversion. Analyze sample according to “Analysis using Gamma Spectroscopy” below.

Analysis using Gamma Spectroscopy. In one method, a high-purity germanium (HPGe) detector and Apex-Gamma with Guard software may suitably be used. Place the sample onto the high-purity germanium (HPGe) detector, after thoroughly cleaning the outside of the vial, at the selected geometry, which includes the position of the vial relative to the detector crystal, the type and shape of the vial itself, and the volume of the liquid inside the vial. All aspects of the geometry must be consistent for the selected efficiency calibration of the HPGe detector, the daily (calibration) suitability check, the radioactive samples, and the blank vial. Launch the Apex-Gamma with Guard software and start the acquisition of data.

Inspect the spectrum and report for any unidentified peaks. Unidentified peaks are expected as analysis times increase due to Compton scattering effects, X-rays as a result of electron capture, or backscattering of gamma rays via the lead shield (unavoidable) (these peaks are mainly visible in low energy regions of the spectrum). Summing effects from lower energy peaks may be seen as well (these peaks are mainly visible in high energy regions of the spectrum). Peaks that are not identified by the software and do not have a tolerance nuclide suggested are not evaluated because the nuclide library includes all possible contaminant nuclides that may present. If a tolerance nuclide is suggested, the software is unable to quantify the nuclide due to a low number of counts. The peak at 511 keV is expected as a consequence of pair production due to positron (positive electron) emission via beta decay, which carries an energy of 1.022 MeV. As a result of the interaction of a positron with an electron somewhere in the sample well, pair production results, which is the annihilation of a positron and electron resulting in two 511 keV photons that separate 1800 from each other due to conservation of energy. Pair production occurs from positron decay of a radionuclide, pair production in the shielding by higher-energy gamma-rays from the source, and pair production in the shielding by high-energy cosmic rays. The presence of the 511 keV peak in blanks is expected due to outside gamma-ray sources, but large 511 keV peaks (>100 counts) could indicate interior contamination in the HPGe-MCA sample well and must be cleaned.

Acceptance criteria. Final dead time of detector <15%. No reported nuclides in blank assays. ¹⁷⁷Lu gamma emission spectra at 113±1 keV and 208±1 keV are present. Replicate Error Ratio for ¹⁷⁷Lu concentrations≤1.96. Lu-177 Purity: NLT 99.9%. Yb-175 Impurity: NMT 0.1%. ^{177m}Lu Impurity: NMT 0.07%. Total Other Impurities: NMT 0.01%.

### Gamma Spectrophotometer

A Mirion/Canberra high purity germanium gamma-ray detector gamma spectrophotometer may suitably be used for qualitative analysis of gamma-ray sources and generate a quantitative spectrum measurement. The Mirion/Canberra spectrophotometer has a photon detection range from approximately 5 keV to 3000 keV. The detector is calibrated for energy and efficiency over the range of 88 keV to 1836 keV using a NIST certified multigamma standard.

### RAC Measurement

RAC measurement may be done using a dose calibrator. An example of a suitable dose calibrator is a CRC®-15R dose calibrator (available from Capintec, United States), a CRC®-ultra dose calibrator (available from Capintec, United States), a Curiementor® 4 isotope calibrator (available from PTW-Frieberg, Germany), an IBC NM dose calibrator (available from Comecer), and an IBC Lite dosage calibrator (available from Comecer). In the assay, for each test vial, 4.5 mL of Omni-Trace water is added to an empty 10 mL glass tubing vial. 0.5 mL of ¹⁷⁷Lu-DOTA-TATE is added to a first vial. 0.5 mL of ¹⁷⁷Lu-DOTA-TATE is added to a second vial. The radioactivity of the samples is assayed using a dose calibrator with the tubing vial dial setting (467*10) and two measurements are obtained per vial. Radioactivity at calibration (RAC) was calculated as follows where “RAA” refers to Radioactivity at Assay:

$$R A C = R A A ( mCi ) \times {e}^{( - ( CAL date and time - ASSAY date and time ) \times ( ln ( 2 ) / 6.647 days )} ) .$$

### Apparent Molar Activity

Apparent molar activity may be calculated from the below equation using the total DOTA-TATE and related substances concentration from the DOTA-TATE Assay and Radiochemical Purity and the free lutetium from the free lutetium assay. Determination of Free Lutetium in ¹⁷⁷Lu DOTA-TATE Injection is determined by Thin Layer Chromatography, and the radioactive concentration is determined from QC.0385 Radioactive Concentration of ¹⁷⁷Lu-DOTA-TATE. In the equation below “AMA” refers to Apparent Molar Activity, “RAC” refers to radioactive concentration, and “TDRS” refers to Total DOTA-TATE and Related Substances:

$$A M A = [ \text{} ( RAC ( mCi / mL ) \times ( ( 100 - Free Lu - 177 ) / 100 ) \times ( 1435.6 ( ng / nmol ) ) ] \text{} / [ ( TDRS ( μg / mL ) ) \times ( 1000 ( ng / μg ) ) ] .$$

### Total Radioactivity Measurement, Content Uniformity, Total Radioactivity, and Filter Integrity

Total radioactivity measurement, content uniformity, total radioactivity, and filter integrity may be done according to the following method.

Content uniformity calculations may be done by the following order to steps. Report the vial number and actual activity at Drug Product Calibration Time. Calculate the average activity at Drug Product Calibration Time from all dispensed vials. Subtract the average actual activity at Drug Product Calibration Time from the actual activity at Drug Product Calibration Time for each vial. Calculate the square of the difference for each vial by squaring each difference value. Sum all the square differences. Calculate the standard deviation by dividing the sum all the square differences value by the number of vials, and then take the square root of the value as indicated in the following equation where “SD” refers to standard deviation:

$$SD = √ [ ( Sum of Squared Differences ) / ( number of vials ) ] .$$

Calculate the content uniformity (relative standard deviation) by dividing the calculated standard deviation by the average actual activity at Drug Product Calibration Time and then multiplying by 100% as indicated in the following equation:

$$Content Uniformity = ( ( SD ) / ( Average ) ) * 100 \% .$$

Fill volume is measured as follows. Remove the stopper from the vial, leaving the product vial in the sample safe. Using a 10 mL disposable plastic syringe and dilator needle, withdraw the ¹⁷⁷Lu-DOTA-TATE product from the vial that is still in the sample safe. Weigh the emptied vial/stopper and record the weight. To determine the Gravimetric Fill Volume, calculate the volume of solution removed using the following equation where “GFV” refers to gravimetric fill volume:

$$G F V = [ filled vial ( g ) - empty vial ( g ) ] / [ density of product ( g / mL ]$$

Filter integrity may be evaluated by a bubble point test. Bubble point tests for evaluating microporous membranes are known in the art and involve measuring the pressure to force either the first air bubble out of a fully wetted phase inversion membrane (the initial Bubble Point, or “IBP”), and the higher pressure which forces air out of the majority of pores all over the phase inversion membrane (foam-all-over-point or “FAOP”). The Bubble Point test is based on the fact that the liquid is retained in the pores of the filter by the surface tension and capillary forces. Methods for conducting initial bubble point and FAOP tests are discussed in U.S. Pat. No. 4,645,602, the disclosure of which is herein incorporated by reference. Procedures for the initial bubble point test and the mean flow pore tests are explained in detail, for example, in ASTM F316-70 and ANS/ASTM F316-70 (Reapproved 1976), which are incorporated herein by reference.

Acceptance criteria: Content uniformity≤3%; Total radioactivity per vial=222 to 272 mCi/vial at Drug Product Calibration Time; Fill volume between 23.0 mL and 25.0 mL; and filter integrity test of ≥3.2 bar.

Free (unbound)¹⁷⁷Lu may be determined using thin layer chromatography (TLC).

TLC Strip Preparation. At a preparation facility humidity of less than 60%, cut iTLC strips 1.5 cm wide by 10-12 cm long using a paper guillotine. iTLC refers to instant thin layer chromatography medium. iTLC silica gel strips may suitably be used and are commercially available, such as from Agilent and Merck. With a ruler, measure 1 cm and 9 cm from the bottom of the strip, respectively, and mark with fine point marker: this will be the origin and solvent front. The iTLC strips are placed in a 100° C. oven for at least 1 hour prior to testing. However, approximately 10 minutes before spotting, remove strips from the oven and allow to cool to room temperature in hood or on benchtop.

Mobile phase. The mobile phase is methanol:water:ammonium hydroxide (20:20:1 v/v/v).

Sample Analysis. Prepare a development chamber by placing about 3 mL of the mobile phase into a TLC developing tank with cylindrical tank lid, or a 100 mL Digitube covered by a 500 mL beaker. Allow the chamber to equilibrate for at least 10 minutes prior to testing. Pipette a 5 μL spot of sample (or 10 μL if performing expiry testing) at the origin of the strip. Place the strip into the development chamber, so that the bottom of the strip is in the solution, but the spot is not submerged. Develop the strip until the solvent front reaches the marking on the strip. Remove the strip from the chamber and allow it to air dry until the strip is completely dry (approximately 20 minutes). Place the strip on the SCAN-RAM and analyze the strip using the instrument parameters listed in the table below.

TLC Instrument Parameters

| Method | Max Time | 0 | sec |
|---|---|---|---|
| Information | Max Count | 0 | |
| | Length | 130.0 | mm |
| | Scan Speed | 1 | mm/sec |
| Channel | Detector | Front | |
| Parameters | Units | Count | |
| | Dwell | 0.2 | sec |
| | Efficiency | 100% | |
| | Lower Limit | 200 | |
| | Upper Limit | 1000 | |
| | High Voltage | 900 | volts |
| Integration | Origin | 10.0 | mm |
| Parameters | Solvent Front | 90.0 | mm |
| | Integration Start | 0.0 | mm |
| | Integration End | 130.0 | mm |

| | | |
|---|---|---|
| Evaluation | Peak Min Area Of | Total Area |
| Parameters | Peak Limit Units | CPM |
| | Default Peak Type | Baseline |

¹⁷⁷LuCl₃ Metal Impurities, Cu, Fe, Pb, Zn and Yb Concentration Measurement

The concentration of metallic impurities in ¹⁷⁷LuCl₃ consisting of Cu, Fe, Pb, Zn and Yb may be measured according to the following inductively coupled plasma mass spectrometry (ICP-MS) method.

An example of a suitable ICP-MS device is a Perkin Elmer NexION 350×ICP-MS.

The following standards were prepared. Standard matrix: 1% HNO₃ and 0.02% HCl. The standard matrix is also used as a blank. ¹⁷⁷Lu Chloride Internal Standard (1 ppm Germanium and 1 ppm Rhenium in 1% HNO₃/0.02% HCl).

A 2.5 ppm Mixed Stock Multi Element (ME) Standard Solution (MSS) was prepared as follows. Transfer 0.125 mL of 1000 ppm Cu, 0.125 mL of 1000 ppm Fe, 0.125 mL of the 1000 ppm Lu, 0.125 mL of the 1000 ppm Pb, 0.125 mL of 1000 ppm Yb, 0.125 mL of 1000 ppm Zn single element standards into the same 50 mL conical tube containing approximately 25 mL of Standard Matrix (1% Nitric Acid/0.02% HCl). Dilute to a final volume of 50 mL with Standard Matrix.

Linearity Standard Preparation. Transfer the amount of 2.5 ppm MSS as indicated as follows into a 50 mL conical tube containing about 25 mL of Standard Matrix (1% Nitric Acid/0.02% HCl) for each linearity standard: 3 ppb standard concentration=0.060 mL of 2.5 ppm MSS; 10 ppb standard concentration=0.200 mL of 2.5 ppm MSS; and 100 ppb standard concentration=2.000 mL of 2.5 ppm MSS. Dilute to a final volume of 50 mL with Standard Matrix.

ICP-MS analysis may suitably be done by the following sequence of steps. Rinse. Calibration Standards: Blank, 3 ppb LuCl₃ Standard, 10 ppb LuCl₃ Standard, 100 ppb LuCl₃ Standard. Rinse. Calibration suitability: 8 ppb ME Standard, 25 ppb ME Standard. Rinse. LuCl₃ Sample. Rinse. Calibration suitability: 25 ppb ME Standard. Print the suitability and sample reports and report the results for Fe, Cu, Zn, Lu, Yb, and Pb.

### Example 1

Comparison of ¹⁷⁷Lu-DOTA-TATE SSTR2 binding of a composition of the disclosure versus a prior art composition.

The somatostatin receptor 2 (SSTR2) binding of two ¹⁷⁷Lu-DOTA-TATE compositions of the disclosure, a prior art ¹⁷⁷Lu-DOTA-TATE composition, and a ¹⁷⁷Lu-DOTA-TATE composition similar to the prior art composition were compared.

The comparative internalization evaluations for the compositions of the disclosure containing (1.5% v/v and 2.5 v/v/% ethanol, respectively) and the prior art compositions occurred on the same day the formulations were assayed at the same time.

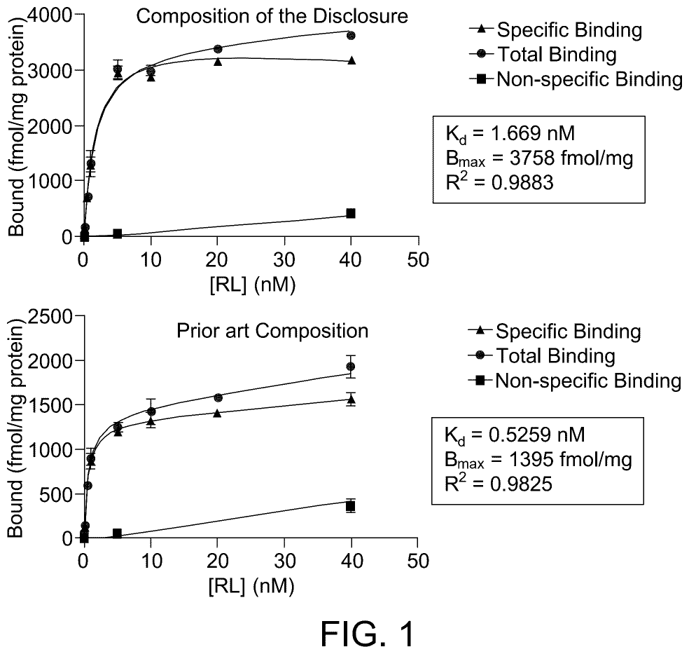

The saturation binding assay showed that the 1.5 v/v % ethanol formulation of the disclosure had a K_{d} of 1.669 nM and a Bₘₐₓ of 3758 fmol/mg, while the prior art formulation had a K_{d} of 0.5259 nM and a Bₘₐₓ of 1395 fmol/mg. The internalization assay revealed that the formulation of the disclosure containing 1.5 v/v % ethanol had a specific internalized activity of 1018-2345 fmol/mg and a percent internalized activity of 87%-91%. The results are depicted in FIG. 1.

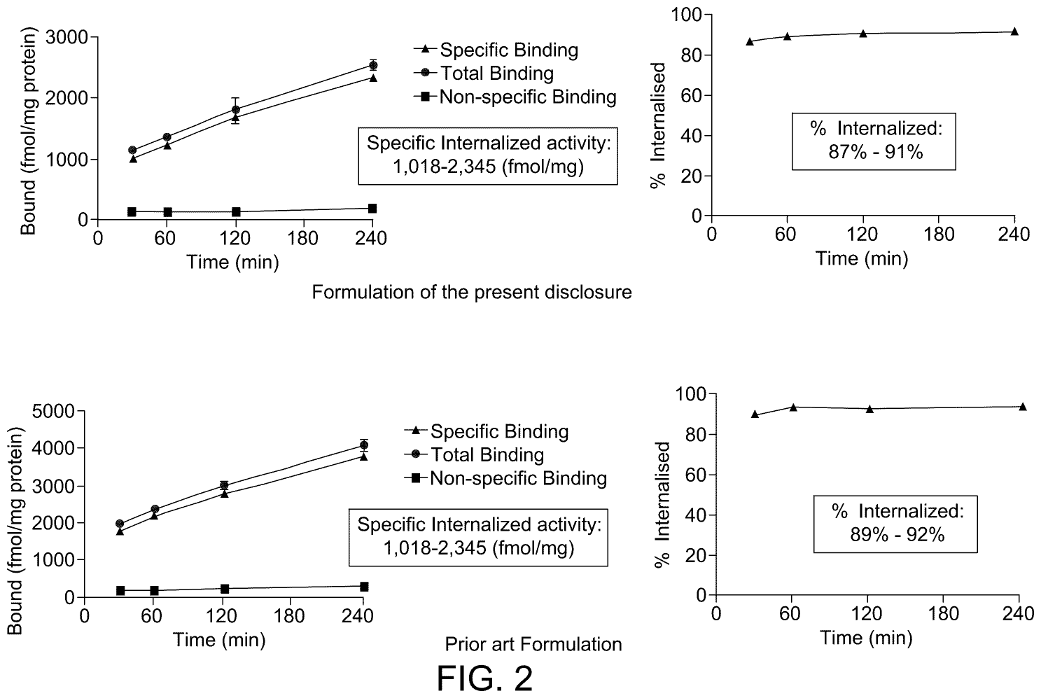

The prior art formulation showed a specific internalized activity of 1796-3774 fmol/mg and a percent internalized activity of 89%-92%. The results are depicted in FIG. 2.

Based on the data presented, the similarities in receptor binding characteristics resulted in equivalent biological activity. The composition of the disclosure and the prior art composition both gave high uptake levels into human-cloned cells expressing SSTR2 receptors. The uptake of radioactivity into cancer cells that express SSTR2 receptors accounts for the therapeutic effect of these agents. The similarities in cellular internalization indicate that the formulation of the disclosure and the prior art composition would have a comparable impact on clinical performance.

An evaluation was done to compare the binding and internalization of a compound of the disclosure comprising 2.5 v/v % ethanol to a composition that replicated the prior art composition. The results from the study are summarized in Table 1 below and confirm that the levels of ethanol tested (0, 1.5 and 2.5%) did not affect the binding affinity (K_{d} and Bₘₐₓ) to SSTR2 receptors or the internalization of radioactivity into the human cloned cells with SSTR2 receptors. Due to non-availability of the prior art formulation, a direct comparison between the formulation of the disclosure (2.5 v/v % ethanol) and the prior art formulation was not performed. The comparability of the 2.5 v/v % ethanol formulation of the disclosure to the prior art formulation was established through the formulation of the disclosure (1.5 v/v % ethanol) which was tested and demonstrated to be comparable to both data sets that were evaluated in real time. Since both the results for the 2.5 v/v % ethanol formulation of the disclosure and the prepared prior art formulation were each comparable to the 1.5 v/v % ethanol formulation of the disclosure that was evaluated in real-time, it is concluded that the affinity and internalization of the 2.5 v/v % ethanol formulation of the disclosure would be similar to the listed drug.

### Table 1

Summary of SSTR2 receptor affinity and biological activity of formulations of the disclosure comprising 1.5 v/v % and 2.5 v/v % ethanol, respectively, and the prior art formulation. % Internal” refers to percent internalization.

| | K_{d} | Bₘₐₓ | % |
|---|---|---|---|
| Formulation | (nM) | (fmol/mg) | Internal. |
| Disclosure (1.5 v/v % EtOH) | 2.697 | 2848 | 87-91% |
| Disclosure (2.5 v/v % EtOH) | 2.859 | 3057 | 85-92% |
| Prior art formulation | 3.956 | 2992 | 82-93% |

### Example 2

Physiochemical testing was done to compare the prior art LUTATHERA® composition versus the compound of the disclosure comprising 1.5 v/v % ethanol and 2.5 v/v % ethanol. The results are reported in Tables 2.1 and 2.2 below. “N/A” refers to not tested.

TABLE 2.1

| Attribute | Prior Art | 1.5 v/v % | Comment |
|---|---|---|---|
| Appearance | Clear, | Clear, | Equivalent |
| | colorless | colorless | |
| pH | 5.27 | 5.23 | Equivalent |
| Osmolality (mOsm/kg) | 284 | 155 | Not Equivalent |
| Radiochemical purity (%) | 98.3 | 98.2 | Equivalent |
| Radionuclidic purity (%) | 100 | 100 | Equivalent |
| Radioactivity Concentration | 6.71 | 6.90 | Equivalent |
| (mCi/mL)-Method 1 | | | |
| Radioactivity Concentration | 8.31 | 8.26 | Equivalent |
| (mCi/mL)-Method 2 | | | |
| DOTA-TATE and related | 7.43 | 8.74 | Not Equivalent |
| Substances (μg/mL) | | | |
| Free (unbound) [¹⁷⁷Lu]Lu³⁺ (%) | 0 | 0 | Not Equivalent |

TABLE 2.2

| Attribute | Prior Art | 2.5 v/v % | Comment |
|---|---|---|---|
| Appearance | Clear, | Clear, | Equivalent |
| | colorless | colorless | |
| pH | 5.27 | 5.25 | Equivalent |
| Osmolality (mOsm/kg) | 284 | 155 | Not Equivalent |
| Radiochemical purity (%) | 98.3 | 99.6 | Equivalent |
| Radionuclidic purity (%) | 100 | N/A | N/A |
| Attribute | Prior Art | 2.5 v/v % | Comment |
| Radioactivity Concentration | 6.71 | N/A | N/A |
| (mCi/mL)-Method 1 | | | |
| Radioactivity Concentration | 8.31 | 10.95 | Not Equivalent |
| (mCi/mL)-Method 2 | | | |
| DOTA-TATE and related | 7.43 | 11.39 | Not Equivalent |
| Substances (μg/mL) | | | |
| Free (unbound) [¹⁷⁷Lu]Lu³⁺ (%) | 0 | 0 | Equivalent |

Osmolality values for the 1.5 v/v % and 2.5 v/v % ethanol compositions of the disclosure are lower than the LUTATHERA® listed drug (155 mOsm/kg versus 284 mOsm/kg), which is considered isotonic. This is not expected to be an issue with respect to the safety or efficacy of the drug since first, it is recognized that the osmolality of drug formulations can vary. For drug formulations intended for intravenous or intravascular injection, the recommended upper osmolality limit should not be more than 1000 mOsm/kg for small-volume injections (i.e., ≤100 mL) for adults. See Wang W., Tolerability of hypertonic injectables, Int J Pharm. 2015; 490(1-2):308-315, the contents of which are incorporated herein in its entirety. Secondly, the drug formulation is infused using sodium chloride which will help to improve the overall tonicity of the solution throughout the patient administration. Since the ¹⁷⁷Lu-DOTA-TATE formulations of the present disclosure are believed to be well below this critical threshold and is infused using sodium chloride, the risk of patient impact is considered low.

The assay results for DOTA-TATE and related substances were different between the 1.5 v/v % and 2.5 v/v % ethanol compositions of the disclosure as compared to LUTATHERA®. Based on the 2.5 v/v % ethanol composition of the disclosure, the target DOTA-TATE content for LUTATHERA® is 250 μg per patient dose. The composition of the disclosure met the proposed specification for DOTA-TATE and related substances (200-300 μg/dose), while LUTATHERA® was below, containing 185 μg of DOTA-TATE and related substances. Although the target for LUTATHERA® is 250 μg per dose, it is believed that based on literature, the tolerance or range established is unknown. It is further believed that the data suggest that LUTATHERA® has a broader tolerance than the ±20% associated with the compositions of the disclosure. The quantity of DOTA-TATE and related substances was shown in the present examples to not impact cell binding, therefore it is believed that this difference is considered low risk.

The radioactivity concentration (RAC) for the 2.5 v/v % composition of the disclosure was higher at the time of assay than LUTATHERA®. This difference is attributed to the time at which the assay was determined relative to the assigned Drug Product Calibration Time. The sample for the 2.5 v/v % composition of the disclosure was prepared for shipment and subsequent use in the cell binding studies discussed above. The time at which RAC was determined was 72 hours earlier than when the listed drug was measured, relative to its time of manufacture. Using decay correction, as depicted in Table 2.3 below, it is believed that the two samples are equivalent.

TABLE 2.3

| | RAC at time of | RAC at time of Drug Product |
|---|---|---|
| Composition | assay (mCi/mL) | Calibration Time (mCi/mL) |
| 2.5 v/v % EtOH | 10.95 | 8.01 |
| LUTATHERA ® | N/A | 8.31 |

Another difference observed between the two formulations evaluated is the result for free[¹⁷⁷Lu]lutetium. In this instance, the results for both of the 1.5 v/v % and 2.5 v/v % ethanol compositions of the disclosure were lower than LUTATHERA®. It is believed that the difference could be influenced by the specific activity of the ¹⁷⁷LuCl₃ used. The formulations of the disclosure were prepared using non-carrier added ¹⁷⁷LuCl₃, while LUTATHERA® incorporates carrier-added radioisotope. Since there is more lutetium presented in the LUTATHERA® sample, it is believed that there is potential for isotope exchange with non-radioactive isotopes which would influence the results of the test. Regardless of the root-cause, the compositions of the disclosure are equivalent or improved with respect to this attribute.

### Example 3

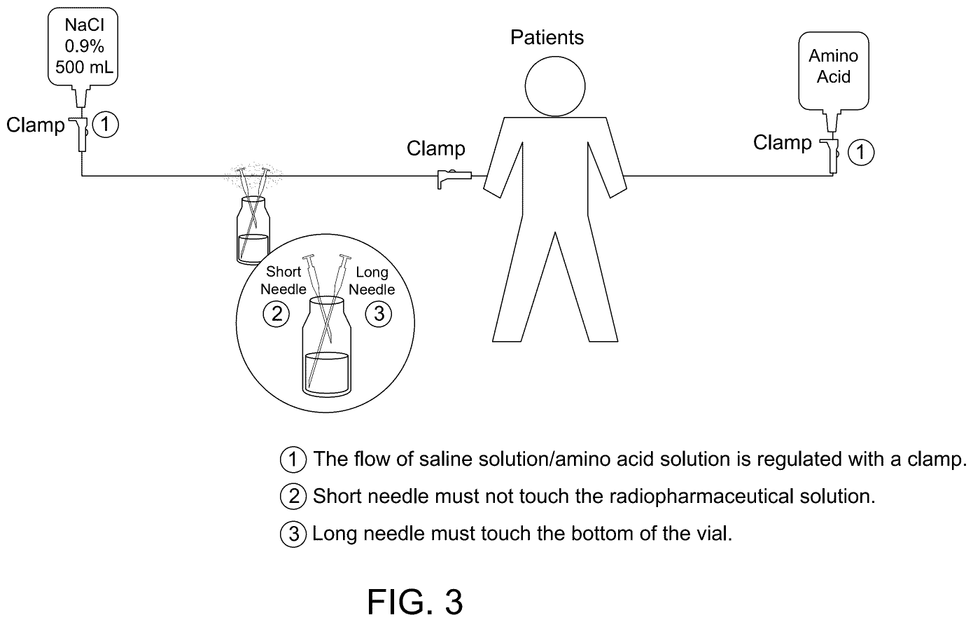

To evaluate the drug formulations as a function of a simulated patient infusion, LUTATHERA® and the 1.5 v/v % composition of the disclosure were each connected to an infusion solution of 0.9% sodium chloride via an IV extension kit fitted with a roller clamp to regulate the flow rate. The infusions were based on the instructions provided in the package insert of the listed drug which is described briefly in FIG. 3. It should be noted that the use of the amino acid solution was not included in the study.

For each infusion, a roller clamp was used to control the introduction of sodium chloride. An initial flow rate of 50 mL/hour to 100 mL/hour was maintained for 5 to 10 minutes and then the flow rate was increased to 200 mL/hour to 300 mL/hour for an additional 25 to 30 minutes.

Samples were taken at specific time points during the simulated infusion (10 min, 20 min, and 40 min). For each sample, RAC, radiochemical purity (RCP), and DOTA-TATE and related substances were evaluated. The results are summarized in Table 3.1 below. In Table 3.1, limit of quantitation (LOQ) for LUTATHERA® was 1.5798 mAu*min, LOQ for the 1.5 v/v % ethanol composition was 1.6890 mAu*min, and “ND” refers to none detected. In both drug formulations evaluated, the radioactivity concentration after 40 minutes was similar. There was some variability in the earlier time points evaluated however it is believed that this could be attributed to slight differences in the flow rate and it is further believed it will not impact the overall efficacy of the drug formulation.

TABLE 3.1

| | Attribute | | | | | |
|---|---|---|---|---|---|---|
| | LUTATHERA ® | | | 1.5 v/v % EtOH | | |
| | Time (minutes) | | | | | |
| | T = 10 | T = 20 | T = 40 | T = 10 | T = 20 | T = 40 |
| RAC (mCi/mL) | 4.64 | 1.07 | 0.036 | 7.92 | 0.147 | 0.023 |
| ¹⁷⁷Lu-DOTA-TATE | 114 | 2.84 | 0.46 | 63.7 | 16.0 | 0.59 |
| (peak area relative to T = 0, %) | | | | | | |
| Radiochemical Impurities Observed | Y | N | N | Y | N | N |
| DOTA-TATE and related | 27.847 | 0.5108 | ND | 18.734 | 3.873 | ND |
| Substances (mAu*min) | | | | | | |

Results for radiochemical purity and DOTA-TATE and related substances were comparable however after the first data point (i.e., T=10 min) data were diluted to a point that the limits of quantitation for both the UV and radiometric detectors was exceeded for impurity peaks. Instead of reporting radiochemical purity as a percentage, the area counts for each sample was reported as a fraction of the T0 sample which was taken pre-infusion.

In both samples, the radioactivity concentration after 40 minutes of infusion was diluted by the saline to less than 1% of the starting RAC. For the DOTA-TATE and related substances assay, a LOQ was established using the peak area for a 0.5 ppm standard. The combined peak area for DOTA-TATE and related substances is reported in Table 3.1 above and suggests that the amount of DOTA-TATE was measured in the simulated infusion solution after 20 minutes was below the LOQ of the LUTATHERA® and was slightly higher than the LOQ in the 1.5 v/v % ethanol composition of the disclosure. No DOTA-TATE or related substances were measured in the simulated infusion from either drug formulation after 40 minutes.

### Example 4

Thirty-two batches of [¹⁷⁷Lu]LuCl₃ were prepared from NCA [¹⁷⁷Lu]Lu³⁺ produced via indirect methods and used to produce drug product during development and tech transfer. The range of the quality attributes are presented in Table 4.1 below.

TABLE 4.1

| | | | Standard |
|---|---|---|---|
| Attribute | Range | Average | Deviation |
| pH | 1.0-1.0 | 1.0 | 0 |
| Radiochemical | Conforms | Conforms | — |
| Identity | | | |
| Radiochemical | 99%-100% | 99.56 | 1 |
| Purity | | | |
| Cu (μg/GBq) | 0.0000-0.0213 | 0.0052 | 0.00879 |
| Fe | 0.0000-0.0901 | 0.0179 | 0.02257 |
| Pb | 0.0000-0.0574 | 0.0151 | 0.01667 |
| Zn | 0.0000-0.2306 | 0.0507 | 0.05224 |
| Yb | 0.0000-0.0335 | 0.0035 | 0.00757 |
| RAC (GBq/mL)^{a} | 0-76 | 63 | 26 |

^{a}RAC was evaluated for 9 of the 32 batches.

From the data summarized in Table 4.1 above, the material attributes of ¹⁷⁷LuCl₃ demonstrate that the manufacturing process is in control and provides material with little to no variability in any material attributes of metallic impurities and radioactive concentration that could potentially influence the RCP of the drug formulation.

### Example 5

Sodium ascorbate was evaluated as a radioprotectant.

The radiochemical yield of the ¹⁷⁷Lu drug substance was evaluated while ranging the concentration of sodium ascorbate present during radiolabeling ranged from 0.3 to 1.2 M (59 to 239 mg/mL). The results from this work are presented in Table 5.1 below, where radiochemical yield is represented as RCP. The data suggest a non-linear fit with the RCP dropping rapidly at higher sodium ascorbate concentrations. To achieve an RCP of at least 95%, the maximum sodium ascorbate concentration that can be used was determined to be approximately 100-110 mg/mL (0.51-0.56 M). In Table 5.1, “[NaAscb]” refers to sodium ascorbate concentration, temperature refers to heat block temperature at the start of test heating, % unbound (free) lutetium [¹⁷⁷Lu]Lu³⁺ was determined by ITLC, and tests 4 and 5 were duplicates.

TABLE 5.1

| | [NaAscb] | [NaAscb] | Temp | Time | RCP | Unbound |
|---|---|---|---|---|---|---|
| Test | in M | in mg/mL | (° C.) | (min) | (%) | [¹⁷⁷Lu]Lu³⁺ |
| 1 | 1.20 | 238 | 94.7 | 20 | 52.6 | 47.4 |
| 2 | 0.90 | 178 | 95.0 | 20 | 83.6 | 16.4 |
| 3 | 0.60 | 119 | 95.2 | 20 | 93.6 | 6.4 |
| 4 | 0.30 | 59 | 95.2 | 20 | 97.6 | 2.4 |
| 5 | 0.30 | 59 | 95.2 | 20 | 98.2 | 1.8 |

A follow-up study was performed to probe further the acceptable range more closely where the effects of the sodium ascorbate concentration on the RCP of the drug substance were examined between 6.3 mg/mL and 77.3 mg/mL while maintaining the amount of radioactivity at time of labeling relatively constant (164-168 mCi). As summarized in Table 5.2, the average RCP for the dataset was 98.15±0.27%. No correlation was observed between the RCP of the drug substance and the concentration of ascorbate in the range evaluated.

TABLE 5.2

| | Radiolabeling | [NaAscb] | RCP |
|---|---|---|---|
| Test | Activity (mCi) | Mg/mL | (%) |
| 1 | 164 | 77.27 | 98.07 |
| 2 | 167.2 | 51.51 | 98.02 |
| 3 | 163.6 | 25.76 | 97.88 |
| 4 | 165.6 | 25.14 | 98.56 |
| 5 | 166.3 | 12.57 | 97.98 |
| 6 | 167.5 | 6.29 | 98.40 |

| | |
|---|---|
| Average | 98.15 |
| % RSD | 0.27 |

### Example 6

Sodium acetate and gentisic acid were evaluated as reaction components for synthesis of ¹⁷⁷Lu-DOTA-TATE as indicated in Table 6.1 below where “NaAcet” refers to sodium acetate and “NaAscb” refers to sodium ascorbate.

TABLE 6.1

| | Reaction Buffer | | | | DOTA- | Heating | |
|---|---|---|---|---|---|---|---|
| Test | NaAcet | NaAscb | Gentisic Acid | [¹⁷⁷Lu]Lu³⁺ | TATE | Time | Temp |
| 1 | 0.15M | None | 5.8 mg/mL | 373 mCi | 257 μg | 15 min | 89° C. |
| 2 | 0.15M | None | 5.8 mg/mL | 380 mCi | 257 μg | 15 min | 91° C. |
| 3 | 0.15M | None | 5.8 mg/mL | 373 mCi | 257 μg | 15 min | 93° C. |
| 4 | 0.15M | 13.7 mg/mL | None | 376 mCi | 257 μg | 15 min | 93° C. |
| 5 | None | 0.2M | None | 220 mCi | 128 μg | 20 min | 95° C. |
| 6 | 0.15M | None | 5.8 mg/mL | 253 mCi | 128 μg | 20 min | 95° C. |

The ¹⁷⁷Lu-DOTA-TATE test compounds in Table 6.2 were formulated with excipients and analyzed for RCP both at time of manufacture (T0) and three days later (T0+3d). The formulations and RCP results are summarized in Table 6.2 below.

TABLE 6.2

| ¹⁷⁷Lu-DOTA- | | RCP | RCP (%, |
|---|---|---|---|
| TATE test | Excipients | (%, T0) | T0 + 3 d) |
| 1 | Na ascorbate (3.3 mg/mL) | 93.66 | 88.91 |
| | Na acetate (1.4 mg/mL) | | |
| | Gentisic acid (0.67 mg/mL) | | |
| | DTPA (0.05 mg/mL) | | |
| | NaCl (6.6 mg/mL) | | |
| 2 | Na ascorbate (3.3 mg/mL) | 94.75 | 94.93 |
| | Na acetate (1.4 mg/mL) | | |
| | Gentisic acid (0.67 mg/mL) | | |
| | Ethanol (1.6%) | | |
| | DTPA (0.05 mg/mL) | | |
| | NaCl (6.6 mg/mL) | | |
| 3 | Na ascorbate (14.8 mg/mL) | 94.86 | 96.37 |
| | Na acetate (1.4 mg/mL) | | |
| | Gentisic acid (0.67 mg/mL) | | |
| | Ethanol (1.6%) | | |
| | DTPA (0.05 mg/mL) | | |
| 4 | Na ascorbate (14.8 mg/mL) | 95.53 | 96.51 |
| | Na acetate (1.4 mg/mL) | | |
| | Ethanol (1.6%) | | |
| | DTPA (0.05 mg/mL) | | |
| 5 | Na ascorbate (10.0 mg/mL) | 98.68 | 96.18 |
| | Ethanol (1.5%) | | |
| | DTPA (0.05 mg/mL) | | |
| 6 | Na ascorbate (3.3 mg/mL) | 96.80 | 94.64 |
| | Na acetate (1.4 mg/mL) | | |
| | Gentisic acid (0.67 mg/mL) | | |
| | DTPA (0.05 mg/mL) | | |
| | NaCl (6.6 mg/mL) | | |

In both data sets, the reactions that were prepared in sodium ascorbate, and subsequently formulated in sodium ascorbate had higher RCP than those that were not.

### Example 7

This example evaluated the use of sodium chloride to enhance the stability of the ¹⁷⁷Lu-DOTA-TATE drug formulations of the disclosure. The radiochemical purity of drug formulations prepared with and without sodium chloride were evaluated along with three ascorbate concentrations (about 10 mg/mL, about 20 mg/mL, and 35 about mg/mL). The data are summarized in Table 7 below where “ND” refers to not determined, “NaAscb vol.” refers to sodium ascorbate volume (mL), “NaAscb cone.” refers to sodium ascorbate concentration (molar, M). All formulations contained 1.5 v/v % ethanol.

TABLE 7

| Test | 1A | 1B | 2A | 2B | 3A | 3C |
|---|---|---|---|---|---|---|
| [¹⁷⁷Lu]Lu³⁺ (mCi) | 162.3 | 197.6 | 141.7 | 194.2 | 155.2 | 136.2 |
| DOTA-TATE (μg) | 255 | 83.5 | 255 | 83.5 | 255 | 255 |
| NaAscb vol. (conc., M) | 4 (0.6) | 1 (0.2) | 4 (0.4) | 1 (0.2) | 4 (0.2) | 1 (0.2) |
| DTPA (mg/mL) | 0.05 | 0.05 | 0.05 | 0 | 0 | 0.05 |
| NaCl (mg/mL) | 1.19 | 0 | 1.19 | 0 | 1.19 | 0 |
| NaAscb (mg/mL) | 31.7 | 36.3 | 21.1 | 22.7 | 10.6 | 10.0 |
| RCP (%, T0) | 98.1 | 97.8 | 98.0 | 97.6 | 97.9 | 98.6 |
| RCP (%, T0 + 3 days) | 97.5 | 97.3 | 97.8 | 97.7 | 96.8 | 98.0 |
| RCP (%, T0 + 7 days) | 97.1 | ND | 97.4 | ND | 96.5 | 96.1 |

From the data presented, there is little difference in radiochemical purity in samples stored for three days and out to seven days post-formulation. T-tests were performed on datasets defined by the approximate concentration of sodium ascorbate present in the formulation (i.e., 10, 20 and 35 mg/mL). All the values from T-tests were greater than 0.05 indicating the differences in the data sets were not significant. Since the presence of sodium chloride does not significantly influence the radiochemical purity of the ¹⁷⁷Lu-DOTA-TATE drug formulation.

### Example 8

The effect of sodium ascorbate on the color of ¹⁷⁷Lu-DOTA-TATE drug formulations of the disclosure was evaluated. It is believed that sodium ascorbate degrades upon exposure to both oxygen and light. Degradation is manifested by a distinct color change from a clear solution that is clear to light yellow in color to a distinct yellow-orange color, depending on the extent of degradation. Drug formulations within the scope of the disclosure containing sodium ascorbate were evaluated for color change at T0, T0+3d, and T0+5d and the results are reported in Table 8 below where each test sample was stored at ambient temperature for the duration of the test.

TABLE 8

| | | Appearance | Appearance |
|---|---|---|---|
| Test | Appearance (T0) | (T0 + 3 d) | (T0 + 5 d) |
| 1 | Clear, colorless | Clear, colorless | Clear, colorless |
| 2 | Clear, colorless | Clear, colorless | Clear, colorless |
| 3 | Clear, colorless | Clear, colorless | Clear, colorless |

### Example 9

The influence of sodium ascorbate concentration on the RCP of ¹⁷⁷Lu-DOTA-TATE drug formulations of the disclosure was evaluated after 3 and 7 days when stored under ambient conditions. The results evaluating the impact of sodium ascorbate concentration on the stability of the drug formulation are summarized in Table 9.1. The results that suggest a minimum concentration of 10 mg/mL of sodium ascorbate is suitable to achieve and maintain the radiochemical purity (RCP) of ¹⁷⁷Lu-DOTA-TATE drug formulation above 95% for at least 3 days.

TABLE 9.1

| Test | 1 | 2 | 3 | 4 | 5 | 6 |
|---|---|---|---|---|---|---|
| [¹⁷⁷Lu]Lu³⁺ (mCi) | 109.3 | 126.2 | 914.0 | 147.4 | 136.2 | 155.2 |
| DOTA-TATE (μg) | 127.5 | 127.5 | 510 | 83.5 | 127.5 | 255 |
| NaAscb vol. (conc.) | 1 (0.05) | 1 (0.1) | 4 (0.4) | 1 (0.2) | 1 (0.2) | 4 (0.2) |
| NaAscb (mg/mL) | 2.5 | 5 | 7.8 | 9.3 | 10 | 10.6 |
| Total Rad/vial (mCi) | 101.6 | 116.0 | 864.0 | 138.5 | 127.0 | 145.5 |
| RCP (%, T0) | 98.4 | 98.0 | 98.0 | 96.2 | 98.6 | 97.9 |
| RCP (%, T0 + 3 days) | 96.1 | 97.0 | 96.8 | 95.6 | 98.0 | 96.8 |
| RCP (%, T0 + 7 days) | 91.2 | 93.9 | ND | 95.6 | 96.8 | 96.5 |
| Test | 7 | 8 | 9 | 10 | 11 | 12 |
| [¹⁷⁷Lu]Lu³⁺ (mCi) | 250 | 141.7 | 197.6 | 162.3 | 194.2 | 165.1 |
| DOTA-TATE (μg) | 250^{a} | 255 | 83.5 | 255 | 83.5 | 83.5 |
| NaAscb vol. (conc.) | (0.2) | 4 (0.4) | 1 (0.2) | 4 (0.6) | 1 (0.2) | 1 (0.2) |
| NaAscb (mg/mL) | 15.0 | 21.1 | 22.7 | 31.7 | 36.3 | 39.6 |
| Total Rad/vial (mCi) | 250.0 | 132.4 | 197.4 | 152.0 | 194.0 | 164.9 |
| RCP (%, T0) | 97.4 | 98.0 | 97.6 | 98.1 | 97.8 | 98.3 |
| RCP (%, T0 + 3 days) | 97.4 | 97.8 | 97.8 | 97.5 | 97.3 | 97.6 |
| RCP (%, T0 + 7 days) | 96.4 | 97.4 | 95.9 | 97.1 | 96.3 | 97.0 |

^{a}Aliquot from 4.72 Ci Reaction

All concentrations evaluated met the proposed specification for radiochemical purity for the drug formulation (i.e., not less than 95%). These data confirm that ascorbate concentration does not impact the RCP of the drug formulation at the concentrations proposed for the drug formulation (i.e., 15 mg/mL).

The effect of reaction buffer ascorbate concentration on radiochemical purity in the radiolabeling reaction was evaluated. Reaction buffers prepared in concentrations between 6.3 and 77.3 mg/mL were evaluated at a constant radiolabeling activity amount. The results obtained for radiolabeling yield are reflected by the initial radiochemical purity results are summarized in Table 9.2. The average RCP for the data was 98.15+/−0.27%. Ultimately there was no correlation with the RCP, and all ascorbate concentrations tested produced drug substance with acceptable radiochemical purity (i.e., ≥95%).

TABLE 9.2

| | Radioactive | Radiolabeling | Initial RCP |
|---|---|---|---|
| | Activity | NaAscorb conc. | (%¹⁷⁷Lu- |
| Sample | (mCi) | (mg/mL) | DOTA-TATE) |
| 1 | 164 | 77.27 | 98.07 |
| 2 | 167.2 | 51.51 | 98.02 |
| 3 | 163.6 | 25.76 | 97.88 |
| 4 | 165.6 | 25.14 | 98.56 |
| 5 | 166.3 | 12.57 | 97.98 |
| 6 | 167.5 | 6.29 | 98.40 |

| | |
|---|---|
| Average | 98.15 |
| % RSD | 0.27% |

The impact of the radioactivity used at time of radiolabeling with sodium ascorbate at a range of 25-31 mg/mL sodium ascorbate concentration in the reaction buffer used during the synthesis of ¹⁷⁷Lu-DOTA-TATE was evaluated. The results are summarized in Table 9.3 and demonstrate that the reaction buffer concentration ranges are suitable to prepare drug substance with up to 9000 mCi at time of radiolabeling.

TABLE 9.3

| | Radioactive | Radiolabeling | Initial RCP |
|---|---|---|---|
| | Activity | NaAscorb conc. | (%¹⁷⁷Lu- |
| Sample | (mCi) | (mg/mL) | DOTA-TATE) |
| 1 | 670 | 25.1 | 98.25 |
| 2 | 240 | 25.1 | 97.40 |
| 3 | 248 | 25.1 | 97.13 |
| 4 | 251 | 25.1 | 97.30 |
| 5 | 254 | 25.1 | 97.59 |
| 6 | 1104 | 25.1 | 97.46 |
| 7 | 1877 | 42.7 | 97.84 |
| 8 | 3937 | 29.0 | 98.88 |
| 9 | 7579 | 31.2 | 97.52 |
| 10 | 8386 | 32.2 | 97.79 |
| 11 | 9107 | 31.6 | 97.21 |
| 12 | 14866 | 29.7 | 99.79 |

| | |
|---|---|
| Average | 97.83 |
| % RSD | 0.80% |

### Example 10

The effect of ethanol content on RCP in ¹⁷⁷Lu-DOTA-TATE drug formulations of the disclosure was evaluated.

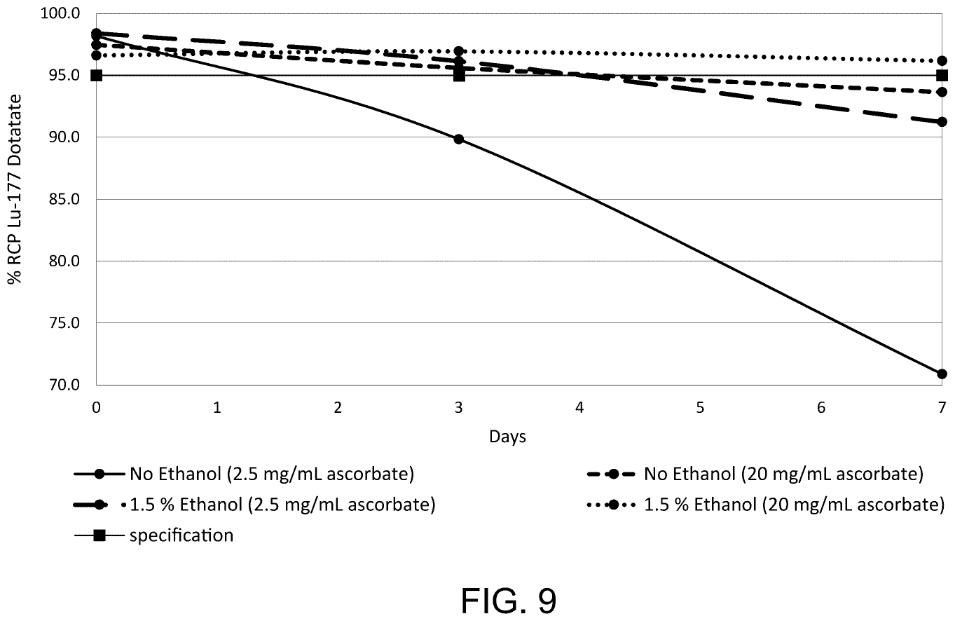

As summarized in FIG. 9, the presence of ethanol has an impact on the radiochemical purity that is influenced by the concentration of sodium ascorbate in the drug formulation. FIG. 9 provides the results of RCP associated with: 2.5 mg/mL ascorbate and no ethanol; 2.5 mg/mL ascorbate and 1.5% ethanol; 20 mg/mL ascorbate and no ethanol; and 20 mg/mL ascorbate and 1.5% ethanol. Details of the individual formulation experiments are provided in Table 10.

TABLE 10

| Lot No. | 1 | 2 | 3 | 4 |
|---|---|---|---|---|
| ¹⁷⁷Lu activity (mCi) | 102 | 132 | 364 | 274 |
| NaAscb (mg/mL) | 2.5 | 2.5 | 20 | 20 |
| Ethanol (v/v %) | 1.5 | 0 | 1.5 | 0 |
| RCP (%, T0) | 98.5 | 98.2 | 96.6 | 97.5 |
| RCP (%, T0 + 3 days)) | 96.1 | 89.8 | 96.9 | 95.6 |
| RCP (%, T0 + 7 days) | 91.2 | 70.9 | 96.2 | 93.6 |

The data in table show that the combination of 1.5 v/v % ethanol and 20 mg/mL sodium ascorbate provides for an RCP at T0+7 days in excess of 95% as compared to the combination of 1.5 v/v % ethanol and 2.5 mg/mL sodium ascorbate which gave an RCP at T0+7 days of 91.2%.

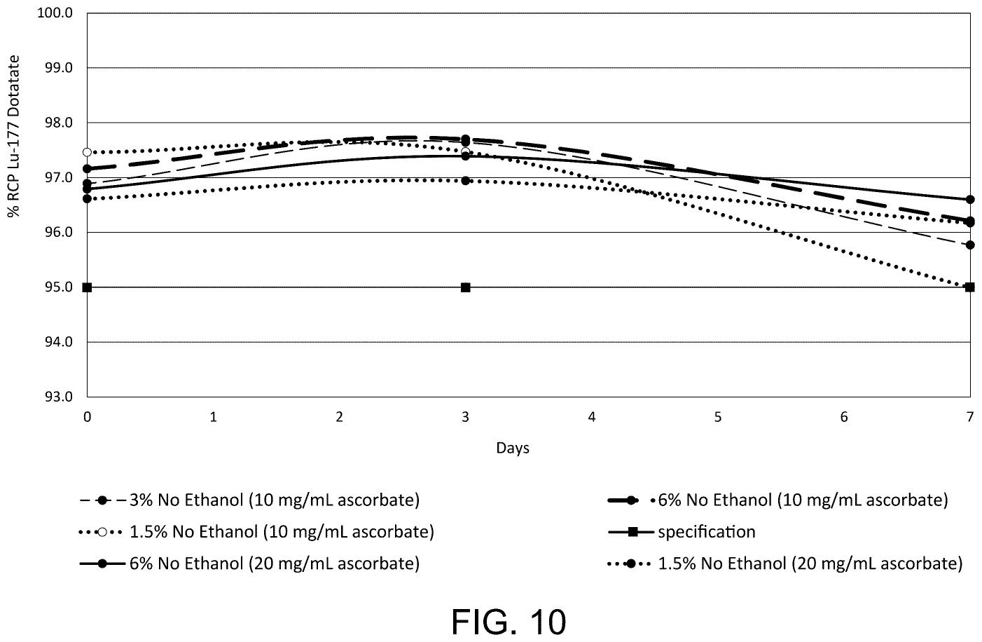

Further evaluations of the combination of ethanol and sodium ascorbate on radiochemical purity of ¹⁷⁷Lu-DOTA-TATE drug formulations as a function of time (0 days, 3 days, and 7 days) were done for the following combinations: 3 v/v % ethanol and 10 mg/mL sodium ascorbate; 1.5 v/v % ethanol and 10 mg/mL sodium ascorbate; 6 v/v % ethanol and 20 mg/mL sodium ascorbate; 6 v/v % ethanol and 10 mg/mL sodium ascorbate; and 1.5 v/v % ethanol and 10 mg/mL sodium ascorbate. The results are depicted in FIG. 10 and indicate that a minimum ethanol concentration of 1.5 v/v % is required to maintain a radiochemical purity at or above 95% for up to 7 days.

### Example 11

The effect of ethanol on ¹⁷⁷Lu-DOTA-TATE drug formulations of the disclosure were evaluated. Ethanol ranging studies were performed with formulations containing 10 mg/mL and 20 mg/mL sodium ascorbate. The results from this evaluation are summarized in Table 11. From the data presented, there is no significant difference in the three levels of ethanol examined in this study and the RCP for each of the drug formulations were all similar at the projected expiry of the drug formulation (3d). The results suggest that over an extended period of time, the presence of ethanol has a positive effect on the stability of the drug formulation and that there is a correlation between increasing amounts of ethanol and higher resulting radiochemical purity values.

TABLE 11

| | | |
|---|---|---|
| Ascorbate | Ethanol | RCP as ¹⁷⁷Lu-DOTA-TATE (%) |

| (mg/mL) | (v/v %) | T0 | T0 + 3 d | T0 + 7 d |
|---|---|---|---|---|
| 10 | 1.5 | 97.5 | 97.5 | 95.0 |
| 10 | 3 | 96.9 | 97.6 | 95.8 |
| 10 | 6 | 97.2 | 97.7 | 96.2 |
| 20 | 6 | 96.8 | 97.4 | 96.6 |
| 20 | 1.5 | 96.6 | 96.9 | 96.2 |

### Example 12

The effect of DTPA on ¹⁷⁷Lu-DOTA-TATE drug formulations of the disclosure was evaluated.

The impact of DTPA was assessed by monitoring the stability of the drug formulation prepared with diluent buffers prepared with DTPA concentration ranging from 0 to 0.15 mg/mL. Radiochemical purity was evaluated at several time points up to the end of the proposed product shelf-life (3 days). The effect of DTPA as an excipient in the drug formulation was evaluated using three (3) formulation variations: (1) DTPA-free, (2) DTPA concentrations ranging between 0.074 and 0.084 mg/mL (n=5), and (3) 0.15 mg/mL DTPA. The results from this study are summarized in Table 12.1.

TABLE 12.1

| | |
|---|---|
| DTPA | RCP (%) |

| Test | (mg/mL) | T0 | T0 + 3 d |
|---|---|---|---|
| 1 | 0 | 98.14 | 96.62 |
| 2 | 0.075 | 97.42 | 96.10 |
| 3 | 0.074 | 97.15 | 96.83 |
| 4 | 0.083 | 96.55 | 95.29 |
| 5 | 0.084 | 95.88 | 95.48 |
| 6 | 0.082 | 97.14 | 96.25 |
| 7 | 0.15 | 97.11 | 97.05 |

The example was repeated for ¹⁷⁷Lu-DOTA-TATE drug formulations of the disclosure at DTPA concentrations over the range of 0.041 to 0.092 mg/mL. 28 total evaluations were done and the DTPA concentrations over that range were determined to have no effect on RCP.

This example was repeated for ¹⁷⁷Lu-DOTA-TATE drug formulations of the disclosure at DTPA concentrations of 0.025 mg/mL (8 total evaluations) and 0.05 mg/mL (4 total evaluations) and free ¹⁷⁷Lu (¹⁷⁷Lu³⁺) was evaluated. The results are shown in Table 12.2 below.

TABLE 12.2

| | | Theoretical | Free | |
|---|---|---|---|---|
| DTPA | | % Free | ¹⁷⁷Lu³⁺ | ¹⁷⁷Lu-DOTA-TATE + |
| (mg/mL) | Test | ¹⁷⁷Lu³⁺ (%) | (%) | ¹⁷⁷Lu DTPA (%) |
| 0.05 | 1 | 0 | 0 | 100 |
| 0.05 | 2 | 4.4 | 0 | 100 |
| 0.05 | 3 | 6.5 | 0 | 100 |
| 0.05 | 4 | 12.1 | 0 | 100 |
| 0.025 | 1 | 0 | 0 | 100 |
| 0.025 | 2 | 5.4 | 0 | 100 |
| 0.025 | 3 | 7.4 | 0 | 100 |
| 0.025 | 4 | 13.0 | 0 | 100 |
| 0.025 | 5 | 0 | 0 | 100 |
| 0.025 | 6 | 2.9 | 0 | 100 |
| 0.025 | 7 | 7.6 | 0 | 100 |
| 0.025 | 8 | 14.1 | 0 | 100 |

From the results summarized above in Table 12.1, there is no correlation between DTPA content and the RCP of the drug formulations of the disclosure. The results in Table 12.2 demonstrate that chelation of the [¹⁷⁷Lu]Lu³⁺ to DTPA is rapid and complete at ambient temperature at DTPA concentrations as low as 0.025 mg/mL in the ¹⁷⁷Lu-DOTA-DATA drug formulations of the present disclosure.

### Example 13

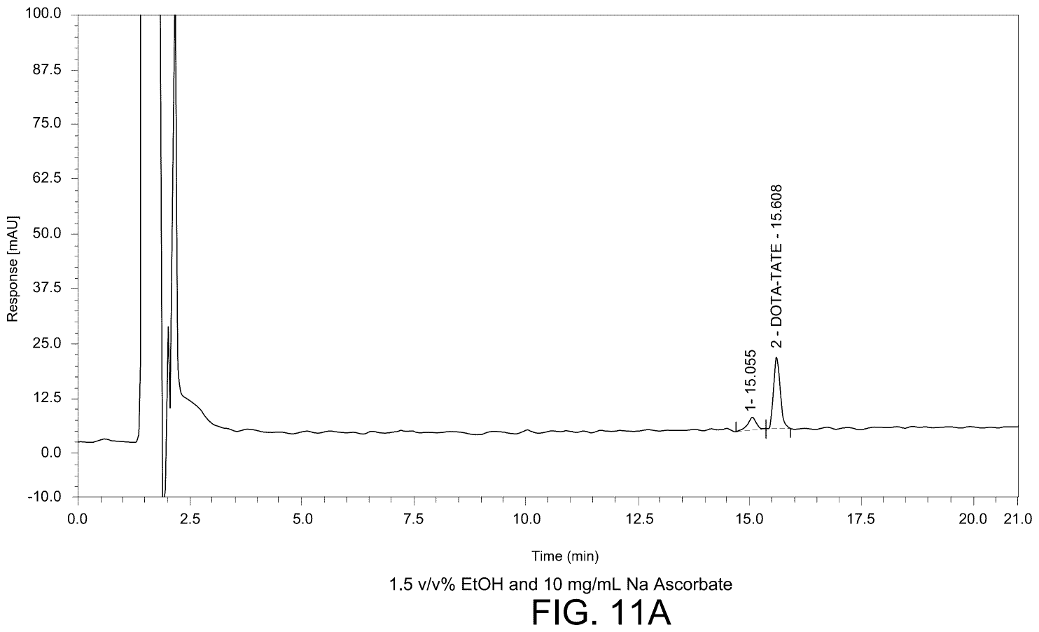

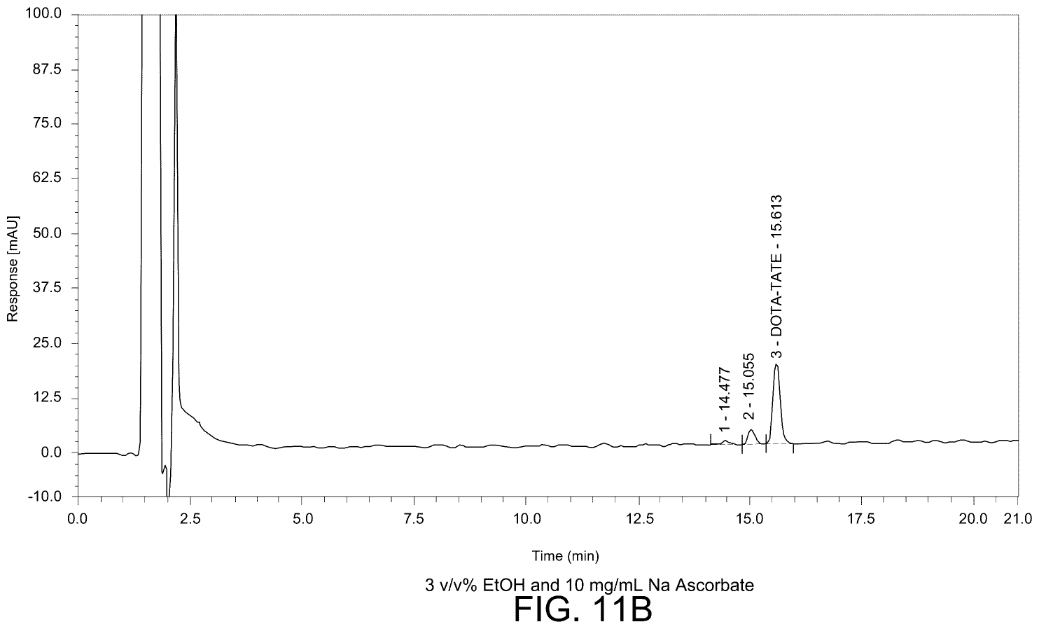

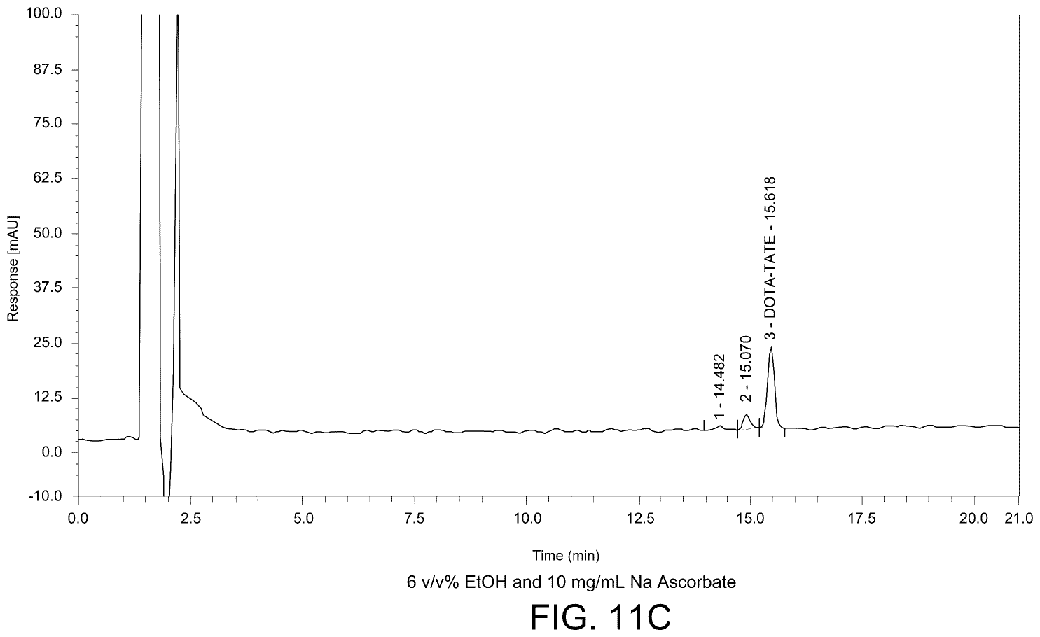

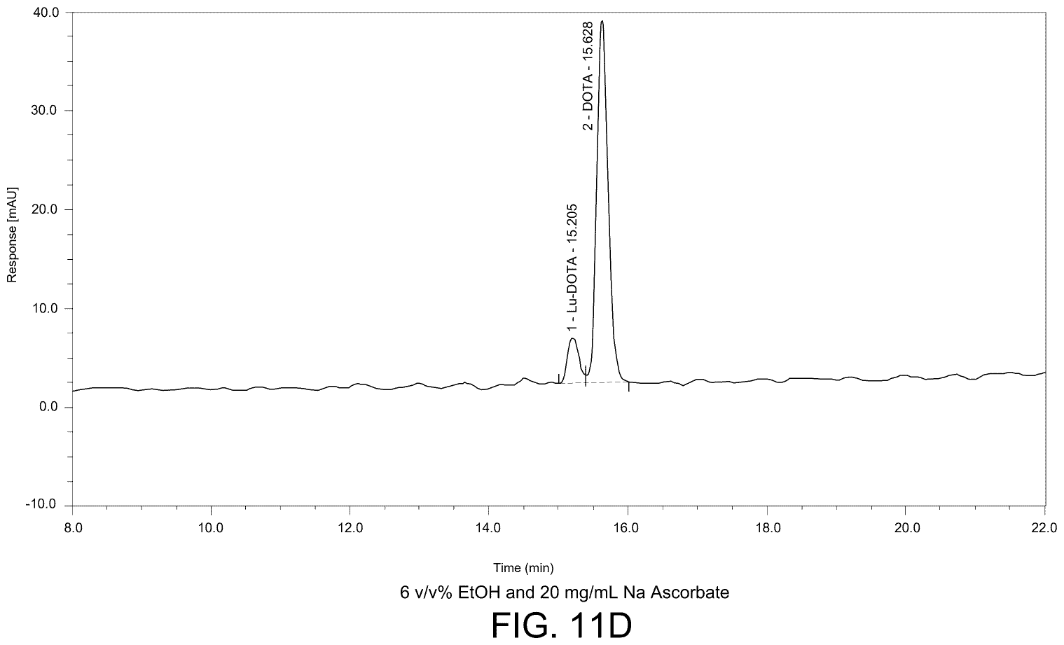

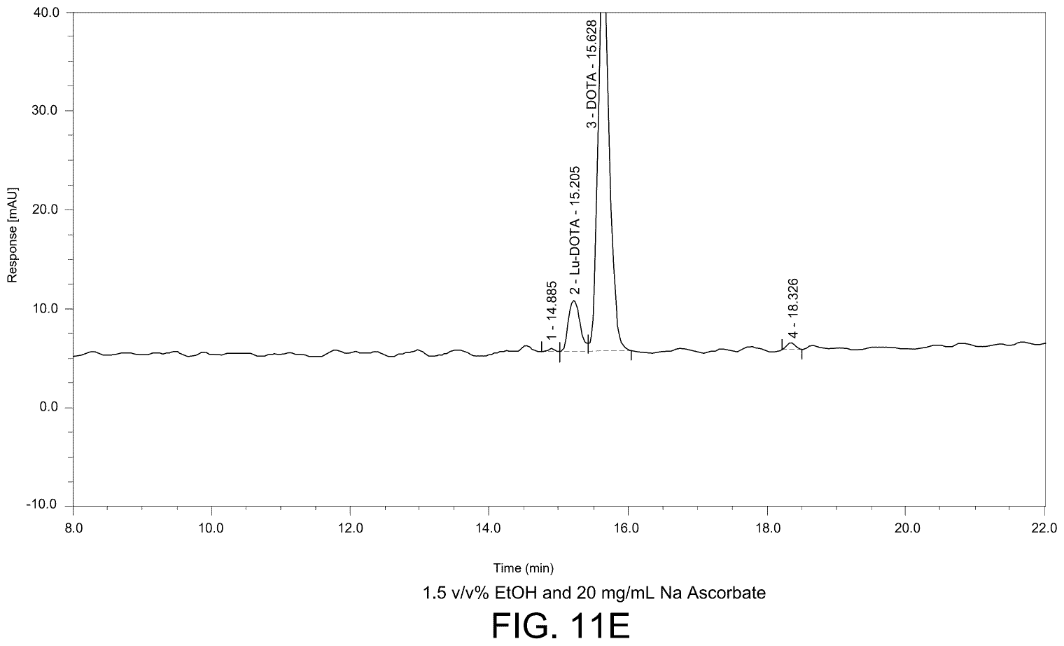

¹⁷⁷Lu-DOTA-TATE formulations of the disclosure were evaluated as a function of increasing ethanol content to evaluate if there is an effect on organic impurities. This was achieved by examining the UV chromatograms obtained using the analytical procedure for radiochemical purity, the assay for DOTA-TATE and related substances, chemical and radiochemical ID and organic impurities. The effects of ethanol on organic impurities were evaluated for: 1.5 v/v % ethanol and 10 mg/mL sodium ascorbate (FIG. 11A); 3 v/v % ethanol and 10 mg/mL sodium ascorbate (FIG. 11B); 6 v/v % ethanol and 10 mg/mL sodium ascorbate (FIG. 11C); 6 v/v % ethanol and 20 mg/mL sodium ascorbate (FIG. 11D); and 1.5 v/v % ethanol and 20 mg/mL sodium ascorbate (FIG. 11E). The results are depicted in FIG. 11A-11E showing the HPLC chromatograms depicting the region of interest around the DOTA-TATE peak (approx. RT: 15 min).

The results indicate that there are no discernable differences in organic impurities as the quantity of ethanol increases. This data supports the conclusion that ethanol over a concentration range of 1.5 v/v % to 6 v/v % does not significantly contribute to organic impurities in the ¹⁷⁷Lu drug formulations of the disclosure.

### Example 14

The effect on RCP for forced (accelerated) degradation studies of two (2)¹⁷⁷Lu-DOTA-TATE drug formulations of the disclosure were done. Evaluated formulations 1 and 2 are summarized in Table 14.1 below.

TABLE 14.1

| Formulation Component | Formulation 1 | Formulation 2 |
|---|---|---|
| DOTA-TATE (μg/mL) | 8 | 10 |
| Na Ascorbate (mg/mL) | 10 | 15 |
| Ethanol (v/v %) | 1.5 | 3.0 |
| DTPA (mg/mL) | 0.075 | 0.05 |

Thermal degradation was done at 55° C. with evaluations done at T0 and T0+3d. Base degradation was done at ambient temperature by basifying the ¹⁷⁷Lu-DOTA-TATE drug formulation to 0.1 N NaOH with evaluations done at T0 and T0+3d. Acid degradation was done at ambient temperature by acidifying the ¹⁷⁷Lu-DOTA-TATE drug formulation with 0.1 N HCl with evaluations done at T0 and T0+3d. Oxidative degradation was done at ambient temperature by adding H₂O₂ to the ¹⁷⁷Lu-DOTA-TATE drug formulation to a final concentration of 3.0 wt. % with evaluations done at T0, T0+1d and T0+2d.

The results for oxidative degradation are present in Table 14.2 below versus a control composition not containing the H₂O₂ oxidizer.

TABLE 14.2

| | | | RCP (%, | RCP (%, |
|---|---|---|---|---|
| Formulation | Sample | RCP (%, T0) | T0 + 1 d) | T0 + 2 d) |
| 1 | Control | 96.0 | ND | 96.7 |
| 1 | Oxidation | 96.0 | ND | 0.1 |
| 2 | Control | 99.7 | 99.6 | 99.3 |
| 2 | Oxidation | 99.7 | 0 | ND |

The results indicate that the drug formulation is not stable under oxidative conditions.

The results for acidic, basic, and thermal degradation are present in Table 14.3 below versus a control composition.

TABLE 14.3

| | | RCP | RCP (%, |
|---|---|---|---|
| Formulation | Sample | (%, T0) | T0 + 3 d) |
| 1 | Control | 98.95 | 96.66 |
| 1 | Thermal | 97.13 | 92.51 |
| 1 | Acid | 98.95 | 77.21 |
| 1 | Base | 98.95 | 17.21 |
| 2 | Control | 99.74 | 99.55 |
| 2 | Thermal (55° C.) | 99.74 | 98.88 |
| 2 | Thermal (−20° C.) | 99.7 | 98.6 |
| 2 | Acid | 99.68 | 85.7 |
| 2 | Base | 99.68 | 10.4 |

The results indicate that acidic and basic conditions impact the RCP of ¹⁷⁷Lu drug formulations of the disclosure. The results further indicate that thermal conditions have a minor impact on RCP.

### Example 15

This example evaluated the effect of radioactivity concentration on the potential degradation of DOTA-TATE and related substances in the ¹⁷⁷Lu-DOTA-TATE drug formulations of the disclosure. The results for two (2) concentration levels of sodium ascorbate (10 mg/mL and 20 mg/mL) are summarized in Table 15.1. Based on the results presented which compares the sum of peak areas corresponding to DOTA-TATE and related substances in the chromatogram, there is not a significant decrease in the DOTA-TATE and related substances assay. Some variability is noted however this variability can be attributed to the slight differences in relative response factors from M-DOTA-TATE complexes and day-to-day variation in instrument response.

TABLE 15.1

| | | | DOTA-TATE | DOTA-TATE |
|---|---|---|---|---|
| | | DOTA-TATE | and related | and related |
| | | and related | substances | substances |
| Na | Radioactivity | substances | peak area | peak area |
| Ascorbate | Concentration | peak area (T0) | (T0 + 3 d) | (T0 + 7 d) |
| 10 mg/mL | 12 (mCi/mL) | 2.881 mAu*min | 2.100 mAu*min | 2.508 mAu*min |
| 10 mg/mL | 13 (mCi/mL) | 3.111 mAu*min | 2.669 mAu*min | 2.626 mAu*min |
| 10 mg/mL | 18 (mCi/mL) | 4.024 mAu*min | 3.669 mAu*min | 3.079 mAu*min |
| 10 mg/mL | 22 (mCi/mL) | 4.959 mAu*min | 3.480 mAu*min | 3.940 mAu*min |
| 20 mg/mL | 19 (mCi/mL) | 7.447 mAu*min | 6.758 mAu*min | ND |
| 20 mg/mL | 19 (mCi/mL) | 6.944 mAu*min | 6.142 mAu*min | ND |

The radiochemical purity of twenty-eight drug products formulated with a radioactivity concentration ranging from 8.1 to 29.1 mCi/mL were monitored for 3 days. A summary of the data is provided in Table 15.2 where tests 1-16 contained 8-11 mg/mL of sodium ascorbate and tests 17-25 contained 19-22 mg/mL sodium ascorbate. Both data sets demonstrate a correlation between radioactivity concentration and radiochemical purity, with the most concentrated samples demonstrating the highest extent of degradation (i.e., lowest RCP values). In Table 15.2 radioactive concentration (RAC) was at the end of the formulation.

TABLE 15.2

| | Sodium | | | RCP |
|---|---|---|---|---|
| | Ascorbate | RAC | RCP | (T0 + |
| Test | (mg/mL) | (mCi/mL) | (T0, %) | 3 d, %) |
| 1 | 8.0-11.0 | 8.10 | 98.56 | 97.98 |
| 2 | 8.0-11.0 | 9.30 | 97.88 | 96.76 |
| 3 | 8.0-11.0 | 9.30 | 97.94 | 97.00 |
| 4 | 8.0-11.0 | 10.10 | 98.21 | 96.45 |
| 5 | 8.0-11.0 | 10.30 | 98.37 | 96.54 |
| 6 | 8.0-11.0 | 10.60 | 98.56 | 97.00 |
| 7 | 8.0-11.0 | 12.10 | 97.40 | 97.02 |
| 8 | 8.0-11.0 | 13.10 | 98.25 | 97.32 |
| 9 | 8.0-11.0 | 13.10 | 98.68 | 96.18 |
| 10 | 8.0-11.0 | 13.40 | 97.59 | 95.59 |
| 11 | 8.0-11.0 | 14.20 | 98.95 | 96.00 |
| 12 | 8.0-11.0 | 16.10 | 98.35 | 97.96 |
| 13 | 8.0-11.0 | 16.20 | 97.46 | 97.47 |
| 14 | 8.0-11.0 | 16.50 | 98.61 | 97.91 |
| 15 | 8.0-11.0 | 17.80 | 97.13 | 95.33 |
| 16 | 8.0-11.0 | 21.80 | 97.30 | 92.49 |
| 17 | 19.0-22.0 | 8.50 | 98.02 | 97.81 |
| 18 | 19.0-22.0 | 16.70 | 98.88 | 97.33 |
| 19 | 19.0-22.0 | 18.10 | 97.14 | 96.25 |
| 29 | 19.0-22.0 | 18.20 | 96.47 | 95.58 |
| 21 | 19.0-22.0 | 18.20 | 96.90 | 95.38 |
| 22 | 19.0-22.0 | 18.40 | 97.15 | 96.83 |
| 23 | 19.0-22.0 | 18.60 | 96.34 | 95.54 |
| 24 | 19.0-22.0 | 18.90 | 97.52 | 95.96 |
| 25 | 19.0-22.0 | 21.40 | 97.79 | 95.52 |

The data in Table 15.2 for test 25 shows that the selection of 19-22 mg/mL sodium ascorbate provided for improved RCP at 3 days as compared to test 16 (8-11 mg/mL sodium ascorbate) for a RAC of about 21.4-21.8 mCi/mL.

### Example 16

The impact of the concentration of the DOTA-TATE intermediate solution on the radiochemical purity of the drug substance prepared using DOTA-TATE ligand prepared at three (3) different concentrations. In all reactions, the ligand to radioisotope ratio was maintained at 6:1 and all reactions were prepared at a representative batch size (i.e., 3-5 Ci at time of radio synthesis). Sample details and results for radiochemical purity are summarized in Table 16 where the average RCP was 97.8±1.02.

TABLE 16

| | DOTA-TATE | Reaction | [¹⁷⁷Lu]Lu-DOTA- |
|---|---|---|---|
| Test | (mg/mL) | scale (Ci) | TATE RCP (%) |
| 1 | 1.00 | 5.1 | 97.7 |
| 2 | 1.40 | 3.8 | 96.8 |
| 3 | 1.50 | 4.3 | 98.9 |

### Example 17

The mole ratio of DOTA-TATE to ¹⁷⁷Lu was evaluated. The drug substance was prepared using DOTA-TATE to ¹⁷⁷Lu³ mole ratios ranging from 2 to 19. The radiochemical purity of the resulting lutetium ¹⁷⁷Lu-DOTA-TATE drug substance was determined as an indirect measure of the radiochemical yield or extent of ¹⁷⁷Lu incorporation. The results are summarized in Table 17 where DT refers to DOTA-TATE and Lu refers to ¹⁷⁷Lu³⁺.

TABLE 17

| | DT:Lu | | |
|---|---|---|---|
| | mole | Radiolabeling | RCP |
| Test | ratio | activity (mCi) | (%) |
| 1 | 19 | 144 | 97.9 |
| 2 | 8.9 | 615 | 98.3 |
| 3 | 7.9 | 123 | 98.3 |
| 4 | 7.3 | 188 | 97.4 |
| 5 | 6.7 | 204 | 97.3 |
| 6 | 6.6 | 207 | 97.1 |
| 7 | 6.5 | 209 | 97.6 |
| 8 | 6.3 | 124 | 98.3 |
| 9 | 5.4 | 180 | 97.8 |
| 10 | 5.3 | 1027 | 97.5 |
| 11 | 5.3 | 183 | 97.6 |
| 12 | 3.9 | 125 | 97.1 |
| 13 | 2.0 | 124 | 67.7 |

The results summarized above indicate that mole ratios of DOTA-TATE to ¹⁷⁷Lu of about four (4) or higher resulted in the average radiochemical yield (RCY) of 97.6% with a relative standard deviation of 0.44%. When lower mole ratios were examined, the radiochemical yield of the resulting product decreased to an unacceptable level of 67.7%. The results suggest that a mole ratio of DOTA-TATE to ¹⁷⁷Lu of at least four (4) provided greater than 95% Lu-177 incorporation. Within this range, the mole ratio of DOTA-TATE to ¹⁷⁷Lu was determined to have a negligible impact on the radiochemical yield of the drug product.

### Example 18

The RCP of ¹⁷⁷Lu-DOTA-TATE was evaluated for reaction temperatures of 80, 90 and 100° C. over a time period ranging between 5, 17.5 and 30 min. The results are presented in Table 18.1 where NaAscb M refers to molar sodium ascorbate concentration with the concentration in mg/mL in parentheses.

TABLE 18.1

| | | NaAscb M | Temp | Time | RCP |
|---|---|---|---|---|---|
| Test | pH | (mg/mL) | (° C.) | (min) | (%) |
| 1 | 4.93 | 0.472 (93.6) | 80 | 5 | 63.26 |
| 2 | 4.06 | 0.472 (93.6) | 80 | 30 | 80.11 |
| 3 | 4.45 | 0.068 (13.4) | 80 | 30 | 98.76 |
| 4 | 3.76 | 0.068 (13.4) | 80 | 5 | 74.91 |
| 5 | 4.46 | 0.270 (53.5) | 90 | 17.5 | 99.47 |
| 6 | 4.46 | 0.270 (53.5) | 90 | 17.5 | 99.27 |
| 7 | 4.07 | 0.472 (93.6) | 100 | 5 | 67.31 |
| 8 | 3.78 | 0.068 (13.4) | 100 | 30 | 99.61 |
| 9 | 4.94 | 0.472 (93.6) | 100 | 30 | 99.46 |
| 10 | 4.48 | 0.068 (13.4) | 100 | 5 | 98.86 |

From the data summarized in Table 18.1, depending on the variables of a particular experiment, all three (3) temperatures evaluated afforded drug substance with radiochemical purity (or radiochemical yield) of approximately 99% at the reaction scale evaluated (i.e., 3 mCi). It should be noted that RCP for the above study was evaluated using iTLC, therefore providing an accurate description of Lu-incorporation but does not provide information on whether or not DOTA-TATE related radiochemical impurities were forming due to thermal degradation.

A wider range of temperatures were evaluated with all other reaction parameters such as buffer pH and concentration fixed. The purpose of this experiment was to evaluate the impact of the reaction temperature during radiolabeling and to determine the appropriate reaction temperature for the process. In this study, the reaction temperatures were at 80° C., 90° C., 95° C. and 120° C. and radioactivity concentrations that ranged from 85.7-373.8 mCi/mL. The results obtained for Lu-177 incorporation are reflected in the initial radiochemical purity results in Table 18.2.

TABLE 18.2

| | Radiolabeling | Temp | Initial |
|---|---|---|---|
| Test | Activity (mCi) | (° C.) | RCP (%) |
| 1 | 221.0 | 80 | 96.60 |
| 2 | 223.0 | 90 | 95.90 |
| 3 | 1111.0 | 95 | 97.46 |
| 4 | 2030.0 | 95 | 97.84 |
| 5 | 132.8 | 95 | 98.30 |
| 6 | 665.0 | 95 | 98.25 |
| 7 | 203.0 | 95 | 97.40 |
| 8 | 224.0 | 95 | 97.13 |
| 9 | 221.0 | 95 | 97.30 |
| 10 | 226.0 | 95 | 97.59 |
| 11 | 147.4 | 120 | 96.15 |

The studies described examined the impact of the reaction temperature on the percent of lutetium complexed with DOTA-TATE, as measured by the radiochemical purity of the drug substance. The results suggest that a reaction temperature of 95° C. provided greater than 97.66% incorporation of ¹⁷⁷LuCl₃ in eight of the tests (Tests 3-10) using radiolabeling activities ranging from 130 mCi to 2000 mCi. Decreasing the reaction temperature to 80-90° C. resulted in slightly lower radiochemical purity average of 96.25% with 1-2% free ¹⁷⁷Lu.

Increasing the reaction temperature to 120° C. resulted in a higher amount of radiolytic degradation which detrimentally impacted the radiochemical purity results obtained (RCP=96.15%). Chromatograms for reactions heated at 80° C., 95° C., and 120° C. indicate that the additional impurities were detected and are more prominent in reactions heated at 120° C. Even though the drug substances prepared at high temperature met specification limit for radiochemical purity of not less than (NLT) 95%, the RCP results are significantly lower (i.e., >1% lower). Based on these data, the optimum heating temperature is about 95° C. but that temperatures as high as about 100° C., 105° C., 110° C., 115° C. or even 120° C. are acceptable.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A parenteral ¹⁷⁷Lu-DOTA-TATE drug formulation comprising:
(1) a therapeutically effective amount of a ¹⁷⁷Lu-DOTA-TATE complex;
(2) a stabilizer component comprising ascorbic acid, or a salt thereof, and ethanol, wherein
(i) the concentration of ascorbic acid or a salt thereof is from 5 mg/mL to 25 mg/mL, and
(ii) the concentration of ethanol is from 1 v/v % to 3 v/v %; and
(3) water.

2. The formulation of claim 1, wherein the radiochemical purity is not less than 95% up to 7 days after formulation thereof.

3. The formulation of claim 1, wherein the therapeutically effective amount of ¹⁷⁷Lu-DOTA-TATE complex is from 222 mCi to 272 mCi at time of drug product calibration.

4. The formulation of claim 1, wherein the ethanol concentration is from 1.0 v/v % to 2.0 v/v %.

5. The formulation of claim 1, wherein the concentration of ascorbic acid or a salt thereof is from 10 mg/mL to 30 mg/mL.

6. The formulation of claim 1 further comprising diethylenetriamine pentaacetic acid (DTPA).

7. The formulation of claim 6, wherein the DTPA concentration is from 0.04 mg/mL to 0.06 mg/mL.

8. The formulation of claim 1, wherein the stabilizer component consists of ascorbic acid, or a salt thereof, and ethanol.

9. The formulation of claim 1 characterized by the absence of sodium chloride.

10. The formulation of claim 1, consisting of
(1)¹⁷⁷Lu-DOTA-TATE complex;
(2) ascorbic acid, or a salt thereof;
(3) ethanol;
(4) DTPA; and
(5) water.

11. The formulation of claim 1, consisting of:
(1) from 222 mCi to 272 mCi of ¹⁷⁷Lu-DOTA-TATE complex at time of drug product calibration;
(2) from 14 mg/mL to 16 mg/mL sodium ascorbate;
(3) 1.5 v/v % or 2.5 v/v % ethanol;
(4) from 0.04 mg/mL to 0.06 mg/mL DTPA; and
(5) water.

12. A method of treating cancers that express somatostatin receptors, the method comprising administering to a subject in need thereof a therapeutically effective amount of the ¹⁷⁷Lu-DOTA-TATE drug formulation of claim 1.

13. The method of claim 12, wherein the cancer is a neuroendocrine cancer.

14. The method of claim 12, wherein the somatostatin receptor is selected from SSTR1, SSTR2, SSTR3, SSTR4, SSTR5, and combinations thereof.

15. The method of claim 14, wherein the somatostatin receptor is SSTR2.

16. The method of claim 12, wherein the therapeutically effective amount of ¹⁷⁷Lu-DOTA-TATE complex is from 222 mCi to 272 mCi at time of drug product calibration.

17. A process for preparing a ¹⁷⁷Lu-DOTA-TATE drug formulation, the process comprising the following order of steps:
(1) prepare ¹⁷⁷Lu-DOTA-TATE in a radiolabeling step comprising the following order of steps
(i) in a reactor combine ¹⁷⁷LuCl₃, DOTA-TATE in solution in a reaction buffer comprising the stabilizer sodium ascorbate, and additional reaction buffer comprising the stabilizer sodium ascorbate,
(ii) preheat the reactor contents to a radiolabeling reaction temperature, and
(iii) react the reactor contents at the radiolabeling reaction temperature for a rection time to form a solution of radiolabeled ¹⁷⁷Lu-DOTA-TATE;
(2) combine the solution of radiolabeled ¹⁷⁷Lu-DOTA-TATE with dilution buffer comprising the stabilizers sodium ascorbate and ethanol and the chelator diethylenetriamine pentaacetic acid (DTPA) to form bulk ¹⁷⁷Lu-DOTA-TATE drug product; and
(3) sterile filter the bulk ¹⁷⁷Lu-DOTA-TATE drug product to form the ¹⁷⁷Lu-DOTA-TATE drug formulation,
wherein the solution of radiolabeled ¹⁷⁷Lu-DOTA-TATE of step (1)(iii) is combined with the dilution buffer in step (2) in the absence of any intervening process steps.

18. The process of claim 17, wherein the radiolabeling reaction temperature is greater than 80° C. and the radiolabeling reaction time at the radiolabeling reaction temperature is at least 10 minutes.

19. The process of claim 17, wherein the ¹⁷⁷Lu-DOTA-TATE drug formulation comprises a therapeutically effective amount of ¹⁷⁷Lu-DOTA-TATE of from 222 mCi to 272 mCi at time of drug product calibration.

20. The process of claim 17, wherein the ¹⁷⁷Lu-DOTA-TATE drug formulation comprises an ethanol concentration of from 1.0 v/v % to 2.0 v/v % and comprises a sodium ascorbate concentration of from 10 mg/mL to 30 mg/mL.
